(19) United States
(12) Patent Application Publication
(10) Pub. No.: US 2003/0054354 A1
(43) Pub. Date: Mar. 20, 2003

(54) Use of antisense oligonucleotide libraries for identifying gene function

(76) Inventors: C. Frank Bennett, Carlsbad, CA (US); Alexander H. Borchers, Encinitas, CA (US); James G. Karras, San Marcos, CA (US)

Correspondence Address: Jane Massey Licata, Licata & Tyrrell P.C., 66 E. Main Street, Marlton, NJ, 08053, US

(21) Appl. No.: 09/938,048

(22) Filed: Aug. 23, 2001

(51) Int. Cl. C12Q 1/68; C07H 21/04

(52) U.S. Cl. 435/6; 536/23.5

(57) ABSTRACT

A method for identifying one or more genes involved in a phenotype of cells, tissues or organisms, comprising the steps of contacting cells, tissues or organisms which exhibit the phenotype with a library of antisense oligonucleotides and performing a primary phenotypic assay to determine which antisense oligonucleotides in the library attenuate the phenotype. These antisense oligonucleotides correspond to genes involved in the phenotype. The method may be used to identify genes involved in various disease states.

### FIELD OF THE INVENTION

The present invention relates to methods for rapidly evaluating the roles of genes in biological processes. More specifically, the invention relates to the use of libraries of antisense compounds, preferably validated antisense oligonucleotides, to determine which gene product or products play a role in determining a particular phenotype.

### BACKGROUND OF THE INVENTION

As reported in Science (291, Feb. 16, 2001), the complete sequence of the human genome has been obtained. However, only a small percentage of genes within the genome have a known function. Many genomics companies have obtained the sequences of portions of the human genome, but have little idea as to the function of the proteins encoded by these new genes. The standard approach to identification of gene function is by random knockout followed by selection of a particular phenotype. This is a very tedious process in which once a particular phenotype has been obtained, extensive experimentation is necessary to determine which gene has been inactivated. In addition, this process has a high failure rate.

Because present approaches used to identify the function of a gene are tedious and have a low success rate, there is a need for a straightforward, accurate method for determining gene function. The present invention addresses this need.

### SUMMARY OF THE INVENTION

One embodiment of the present invention is a method for identifying one or more genes involved in a response by a cell, tissue or organism to a stimulus (e.g., a chemical compound), comprising the steps of: contacting cells, tissues or organisms which are capable of exhibiting a particular response to the stimulus with a library of validated inhibitors of a gene or gene product, preferably validated antisense compounds, more preferably validated antisense oligonucleotides, prior to treatment with the stimulus, each of the oligonucleotides being known to specifically inhibit one molecular target; and determining which antisense oligonucleotides within the library modulate the response to the stimulus, wherein antisense oligonucleotides which modulate the response correspond to gene products involved in the response to the stimulus. Preferably, the cells are divided into one or more substantially identical subpopulations prior to contacting with the library of antisense oligonucleotides, wherein each subpopulation is contacted with one member of the library of antisense oligonucleotides. In one aspect of this preferred embodiment, the compound is a cytokine or growth factor. Advantageously, the stimulus is addition of a cytokine or growth factor. Preferably, the cytokine or growth factor is TNF-α, IL-1 or IFN-γ. In another aspect of this preferred embodiment, the response is secretion of a compound. Advantageously, the compound is a cytokine or growth factor. Preferably, the response is modulation of expression of a cell surface protein. In one aspect of this preferred embodiment, the cell surface protein is a cell adhesion protein. Advantageously, the response is modulation of inflammation. Preferably, the response is inhibition of inflammation. Alternatively, the response is stimulated. In one aspect of this preferred embodiment, the response is a modulation of apoptosis or cell cycle profile. Preferably, the response is modulation of angiogenesis. In another aspect of this preferred embodiment, the response is modulation of insulin signaling, glycogenolysis or adipocyte differentiation.

The present invention also provides a method for identifying one or more genes involved in a phenotype of a cell, tissue or organism, comprising the steps of: contacting one or more substantially identical subpopulations of the cell, tissue or organism which exhibits the phenotype with a library of antisense oligonucleotides; wherein each subpopulation is contacted with one member of the library of antisense oligonucleotides; and performing a primary phenotypic assay to determine which antisense oligonucleotides within the library modulate the phenotype, wherein antisense oligonucleotides which modulate the phenotype correspond to genes involved in the phenotype. Preferably, the phenotype is associated with a disease state. In one aspect of this preferred embodiment, the disease state is cancer, undesired angiogenesis, inflammation or a metabolic disorder. Advantageously, the method further comprises the step of performing a secondary phenotypic assay. Preferably, the secondary phenotypic assay is a low density array. In another aspect of this preferred embodiment, the method further comprises the step of performing a tertiary phenotypic assay. Preferably, the tertiary phenotypic assay is a high density array.

Another embodiment of the present invention is a method for identifying genes expressed in dendritic cells that regulate co-stimulation of T-cells, comprising the steps of: culturing dendritic cells in the presence of one or more cytokines to activate the dendritic cells; contacting one or more substantially identical subpopulations of the activated dendritic cells with a library of antisense oligonucleotides, wherein each subpopulation is contacted with one member of the library; adding T-cells to the antisense oligonucleotide-treated activated dendritic cells; and measuring IL-2 production, wherein antisense oligonucleotides which modulate IL-2 production correspond to genes which play a role in co-stimulation of T cells. Preferably, the cytokines comprise IL-4 and GM-CSF. In one aspect of this preferred embodiment, the method further comprises the step of adding a CTLA4-Ig fusion protein after treatment with antisense oligonucleotide. Advantageously, the antisense oligonucleotide inhibits production of IL-2.

The present invention also provides a method for identifying genes that play a role in T cell-mediated inflammation, comprising the steps of: culturing dendritic cells in the presence of one or more cytokines to activate said dendritic cells; contacting one or more substantially identical subpopulations of the activated dendritic cells with a library of antisense oligonucleotides, wherein each subpopulation is contacted with one member of the library; adding T-cells to the antisense oligonucleotide-treated activated dendritic cells; and measuring IL-2 production, wherein antisense oligonucleotides which inhibit IL-2 production correspond to genes whose products are involved in increasing inflammation. Preferably, the cytokines comprise IL-4 and GM-CSF. In one aspect of this preferred embodiment, the method further comprises the step of adding a CTLA4-Ig fusion protein after treatment with antisense oligonucleotide.

Another embodiment of the present invention is a library comprising between about 10 and 10,000 prevalidated antisense oligonucleotides.

### BRIEF DESCRIPTION OF THE DRAWINGS

FIG. 1 is a schematic diagram showing identification of cancer specific molecular targets using antisense directed synthetic lethality.

FIG. 2 shows the effect of multiple different antisense oligonucleotides on blood glucose levels in the db/db mouse model of Type II diabetes.

FIGS. 3A-3E are graphs showing results of an endothelial cell tube formation assay using a library of antisense oligonucleotides. Higher scores indicate more tube formation. Unt=untreated, lipid=negative control.

FIGS. 4A-4E are graphs showing results of a matrix metalloprotease (MMP) assay using a library of antisesnse oligonucleotides. Higher numbers indicate greater amounts of MMP RNA. UTC=untreated control, lipid=negative control.

FIG. 5 is a graph showing results of a triglyceride assay using a library of antisense oligonucleotides transfected into human preadipocytes which were then treated with a medium which induced differentiation into adipocytes. Triglycerides are accumulated only by differentiated adipocytes. Utc/ud=untreated undifferentiated control cells; utc/d=untreated differentiated control cells. Lower numbers indicate less differentiation.

FIG. 6 is a graph showing results of a leptin assay using a library of antisense oligonucleotides. The media from the cells described in FIG. 5 were assayed for leptin which is only secreted by differentiated adipocytes. Utc/ud=untreated undifferentiated control cells; utc/d=untreated differentiated control cells. Lower numbers indicate less differentiation.

### DETAILED DESCRIPTION OF THE INVENTION

The present invention provides methods for determining the function of a gene in a cell, tissue or organism without the need for gene knockouts and laborious experimentation to determine the identity of the gene whose function was knocked out. The method involves the use of libraries of validated antisense inhibitors of a gene or gene product. These antisense inhibitors include libraries of small molecules, antibodies and oligonucleotides. In a preferred embodiment, the validated inhibitors are antisense oligonucleotides. As used herein, the term “validated” means that the ability of the inhibitor to specifically inhibit expression of a target gene or gene product has been previously confirmed. In a preferred embodiment, a “library” of antisense oligonucleotides is used which contains between about 10 and 10,000 antisense oligonucleotides. As used herein, the term “library” as applied to antisense oligonucleotides is intended to mean any organized collection of such oligonucleotides, such that the systematic assay of all members of the library is possible by manual or automatic means, and in a fashion that allows the testing of a significant number of members of the library in a single step or steps. The oligonucleotide library is screened using various in vitro and in vivo functional assays in which one antisense oligonucleotide is used per condition (i.e., one antisense oligonucleotide per well in a microtiter plate or one antisense oligonucleotide per animal). In a preferred embodiment, the oligonucleotide library is assayed using multi-well plates (e.g., 96- or 384-well) or arrays including gene chips. The plate can be a single plate or multiple plates. Antisense oligonucleotides which modulate a particular response correspond to gene products involved in the response.

Many validated antisense oligonucleotides to particular gene targets have been described, including but not limited to, protein kinase C (U.S. Pat. No. 6,117,847), ras (U.S. Pat. No. 6,117,848), TNF-α (U.S. Pat. No. 6,080,580), raf (U.S. Pat. Nos. 5,952,229 and 6,096,626), survivin (U.S. Pat. Nos. 6,077,709 and 6,165,788), B7 (U.S. Pat. No. 6,077,833), PI3 kinase subunits (U.S. Pat. Nos. 6,165,790, 6,100,090 and 6,133,032), the cell adhesion molecules ICAM-1, ELAM-1 and VCAM-1 (U.S. Pat. Nos. 5,514,788, 5,843,738, 6,093,811 and 6,096,722), MEKK1 (U.S. Pat. No. 6,168,950), BCL-X (U.S. Pat. No. 6,172,216), papillomavirus (U.S. Pat. No. 6,174,870), integrin-linked kinase (U.S. Pat. No. 6,177,273), TNF-α converting enzyme (U.S. Pat. No. 6,180,403), MDM2 (U.S. Pat. No. 6,184,212), cytosolic phosphoenolpyruvate carboxykinase (U.S. Pat. No. 6,187,545), AKT-3 (U.S. Pat. No. 6,187,586), FRA-1 (U.S. Pat. No. 6,124,133), PDK-1 (U.S. Pat. No. 6,124,272), telomeric repeat binding factor 1 (U.S. Pat. No. 6,130,088), JNK proteins (U.S. Pat. No. 6,133,246), IL-5 and IL-5Rα (U.S. Pat. No. 6,136,603), BCL-6 (U.S. Pat. No. 6,140,125), CD44 (U.S. Patent o. 6,150,162) and STAT3 (U.S. Pat. No. 6,159,694). The entire contents of all of the patents mentioned above are incorporated herein by reference. The library may contain any desired antisense oligonucleotide to any target nucleic acid or gene.

The present method can be practiced by contacting cells, tissues, organs or organisms which are capable of exhibiting a particular phenotype with a library of validated antisense compounds, preferably antisense oligonucleotides, to determine which gene or genes is involved in a particular phenotype, disorder or disease. As used herein, the term “library” means between about 10 and 100,000 antisense compounds. The method can also be used to identify one or more genes involved in a response by a cell, tissue or organism to a stimulus by contacting cells, tissues or organisms which exhibit a particular response to the stimulus with a library of antisense compounds prior to treatment with the stimulus and determining which compounds within the library modulate (stimulate or inhibit) the response. As used herein, “stimulus” is any treatment which elicits a response by the cell, tissue or organism and includes, but is not limited to, chemical compounds such as cytokines, growth factors and other signaling molecules, radiation (e.g., x-rays, microwaves, visible light, ultraviolet light), electrical stimulation, changes in temperature, changes in pH, stress (including chemical stressors) and the like. In a preferred embodiment, a cell-based assay is used in which cells are pretreated with a validated antisense oligonucleotide library, followed by measurement of a response of the cells to a compound. Optionally, cells are treated with a first compound which modulates (stimulates or inhibits) production of a second compound, such as a cytokine, after oligonucleotide treatment. Oligonucleotides which modulate the response compared to control oligonucleotides target a gene involved in the response of the cell to the compound. This represents a tremendous advantage over knockout-based methods because once an oligonucleotide is identified which is involved in a particular response or pathway, the identity of the corresponding gene is immediately known because each oligonucleotide is a validated and specific inhibitor of a single gene or gene product. Examples of such assays include an assay to measure functional stimulation of T lymphocyte responsiveness by dendritic cells (DC) and a 96-well microtiter assay to identify novel genes required for activation of keratinocytes. These are described in Examples 10 and 12, respectively.

Therapeutic areas suitable for primary oligonucleotide library-based phenotypic assays include oncology (e.g. synthetic lethality such as apoptosis and cell cycle), angiogenesis, inflammation (e.g. T-cell, endothelial cell and epithelial cell activation and co-stimulation) and metabolism (e.g., promotion of insulin signaling in adipocytes, modulation of adipocyte differentiation and inhibition of glycogenolysis). Primary phenotypic assays allow rapid and high throughput gene functionalization. The high throughput pharmacology “read-out” includes, for example, increased production of cytokines or growth factors (e.g. interleukins, TNF-α, TGF-β, IFN-γ), induction of apoptosis (e.g. caspase-3 activity), changes in cell cycle profile (flow cytometry), decreases in blood glucose levels (e.g., diabetic mouse models), modulation of insulin signaling or glycogenolysis, decreased angiogenesis (e.g. endothelial cell tube formation assay, MMP2 assay), modulation of inflammation and modulation of expression of cell surface proteins.

After gene functionalization has been accomplished using primary phenotypic assays, secondary (low density expression arrays) and tertiary (high density arrays) phenotypic assays may optionally be performed. Secondary phenotypic assays are preferably conducted using low density, high throughput custom arrays. These assays have a sensitivity comparable to Northern blots and PCR (about 0.3 copies per cell), are linear over 3 logs, are highly reproducible, are low density (300-400 genes per slide, duplicate points), are inexpensive and “customizable.” Cell cycle, apoptosis, angiogenesis, inflammation and metabolic arrays are available. Tertiary phenotypic assays include Affymetrix high-density arrays which have a high data content (e.g., 12,600 probe sets for human chip 95a).

Angiogenesis

Angiogenesis, the growth of new blood vessels by endothelial cells, is important in the development of a number of human diseases. In particular, angiogenesis is believed to be important in regulating the growth of solid tumors. Without new vessel formation, it is believed that tumors will not grow beyond a few millimeters in size. Angiogenesis inhibitors also have potential for treatment of diabetic retinopathy, rheumatoid arthritis and psoriasis. In angiogenesis, key genes may be identified using the methods of the present invention that regulate endothelial proliferation and survival. Primary phenotypic assays include endothelial cell tube formation assay, MMP2 activity fluorogenic assay and reverse transcriptase-polymerase chain reaction (RT-PCR) of “angiogenic hallmark genes” such as integrin-β3, endothelial nitric oxide synthase and collagen type III-α1.

MMPs play an important role in angiogenesis by degrading extracellular matrix and allowing endothelial cells to migrate and form new vessels. In the MMP assay, media above antisense oligonucleotide-treated endothelial cells is added to microtiter plates, followed by addition of a substrate which becomes fluorescent after degradation by MMPs. Fluorescence on the plate is read by a fluorescence plate reader after an overnight incubation at 37° C. Inhibition of MMP activity is measured by a decreased in fluorescence.

The formation of tube-like structures in vitro by endothelial cells is believed to recapitulate the process of new blood vessel formation in vivo. In this assay, antisense oligonucleotide-treated endothelial cells are removed from one microtiter plate and placed on a second plate which has been coated with a mixture of extracellular matrix proteins and growth factors. The extracellular matrix proteins promote the formation of tube-like structures.

The angiogenic hallmark genes mentioned above are believed to be important for migration and proliferation of endothelial cells during angiogenesis. In this assay, total RNA is prepared from antisense oligonucleotide-treated endothelial cells in a 96 well plate, split into four fractions and assayed by quantitative real-time PCR (TaqMan) in a 384 well plate for the expression of the three hallmark genes, plus glyceraldehyde 3-phosphate dehydrogenase as a control.

Secondary phenotypic assays include low-density human angiogenesis arrays and migration assays. About 200 genes are believed to play a role in angiogenesis. These genes have been shown to be differentially expressed in normal and tumor endothelium. In the low-density angiogenesis array, RNA is extracted from oligonucleotide-treated cells, reverse transcribed into cDNA, amplified by PCR to make a radiolabeled probe and hybridized to a glass chip with specific oligonucleotides covalently attached. This assay provides expression analysis of 190 genes believed to play a role in angiogenesis.

The ability to migrate through an extracellular matrix is required for endothelial cells to form new blood vessels. Quiescent endothelial cells do not do this, and so the ability to migrate is believed to be a key feature of cells that have developed an angiogenic phenotype.

Tertiary phenotypic assays include high density (Affymetrix) DNA arrays. This allows transcriptional profiling of endothelial cells after specific gene knockdown which will provide insight into individual gene function. In this assay, RNA is extracted from antisense oligonucleotide-treated endothelial cells, and the expression of a large number of genes is determined by array analysis. Examples of phenotypic assays for angiogenesis are summarized in Table 1.

TABLE 1

| Assay name | Target end-point | Cell line |
|---|---|---|
| Matrix | MMP activity | Human umbilical |
| metalloprotease | | vein endothelial |
| (MMP) assay | | cell (HUVEC) |
| Expression of | Integrin-β3, endothelial | HUVEC |
| angiogenic | nitric oxide synthase, | |
| hallmark genes | collagen type III-α1, | |
| | glyceraldehyde 3- | |
| | phosphate dehydrogenase | |
| Tube formation | Tube formation | HUVEC |
| assay | | |
| Low-density DNA | About 200 genes | HUVEC |
| arrays | differentially expressed | |
| | in normal and tumor | |
| | endothelium | |
| Migration assay | Optical density of | HUVEC |
| | migrated cells | |
| High-density | 12,600 human genes | HUVEC |
| affymetrix DNA | | |
| array | | |

Cancer

Cancer assays are designed to identify genes that preferentially regulate the maintenance of the malignant phenotype. The effects of inhibiting gene expression on the induction of cell death, proliferation, differentiation and changes in the cell cycle profile are determined in either normal or malignant cells. Primary phenotypic assays include induction of apoptosis (caspase-3 activity) and cell-cycle profile (flow cytometry)

A relatively early event in apoptosis is the activation of specific proteases, such as caspase-3, that are responsible for the cleavage of several cellular components related to DNA repair and regulation. Cancer cells escape programmed cell death. Thus, the identification of genes that regulate this cellular process is important. In this assay, a cell permeable fluorogenic caspase-3 substrate (e.g., DEVD-AFC) is added to antisense oligonucleotide-treated cells such as MCF7 breast carcinoma cells. Cells undergoing apoptosis have elevated levels of caspase-3 which cleaves the specific substrate, resulting in release of fluorescent AFC that is measured with a fluorometer.

Inhibition of target gene expression with an antisense oligonucleotide can also lead to cell cycle arrest and/or apoptosis. Cell cycle profile assays identify genes that when inhibited will lead to specific, differential effects in tumor versus normal cells. In this assay, antisense oligonucleotide-treated cells are fixed with ethanol, stained with propidium iodide and analyzed on a flow cytometer for cell cycle profiles.

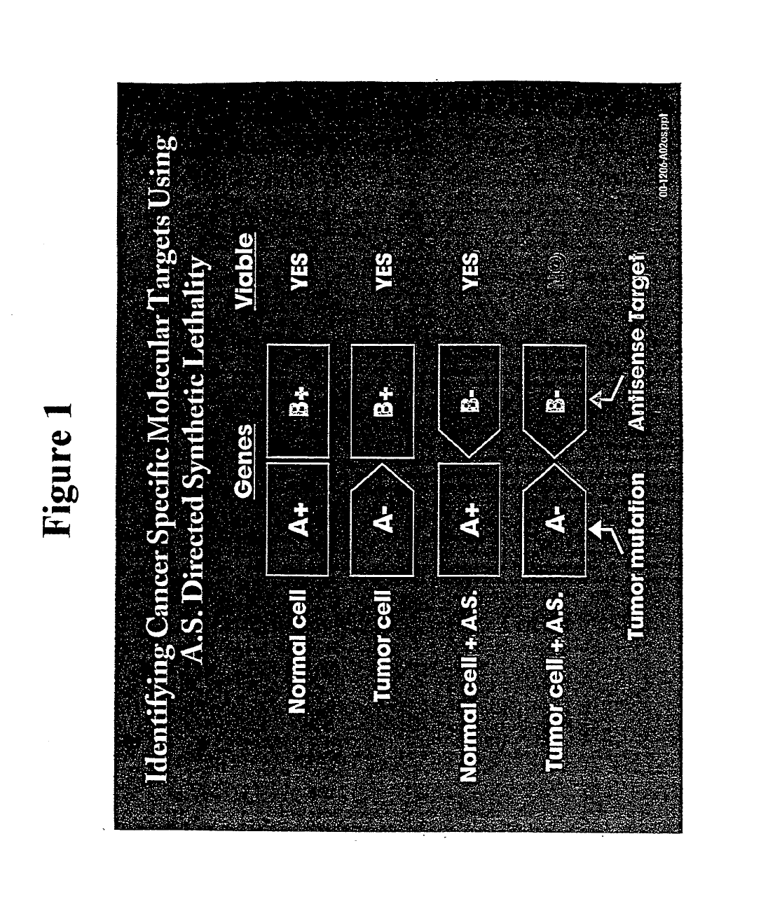

In a preferred embodiment, synthetic lethality assays are used for cancer gene target identification to identify genes that either selectively or non-selectively induce apoptosis or cell-cycle perturbation in tumor compared to normal cells. Selectivity may be based upon either over-expression/activation of a target preferentially in tumor cells (target-driven), or by context in which two genes are synthetic lethal if disruption of either gene is compatible with cell viability but if loss of both genes causes apoptosis (context-driven). Antisense oligonucleotides disrupt one gene, and the genetic background (e.g., mutations and gene deletions) present in the tumor cells are responsible for the other disruption. The target is present in both normal and diseased tissue. However, selectivity is obtained due to transforming mutations in different genes that alter the tumor's requirement for the target protein. Identification of cancer specific molecular targets using antisense directed synthetic lethality is summarized in FIG. 1. In one embodiment, p53 wt and p53 mutant tumor cells are compared to normal, primary cells, as this is one of the most widespread mutations found in human cancers.

Secondary assays include low-density DNA arrays which provides expression analysis of 190 genes believed to play a role in cell cycle/apoptosis. In this assay, RNA is extracted from antisense oligonucleotide-treated cells, reverse transcribed into cDNA, PCR amplified to make a radiolabeled probe and hybridized to a glass chip with specific oligonucleotide covalently attached.

Tertiary assays include high density (Affymetrix) DNA arrays (U95a chip). Transcriptional profiling of tumor versus normal cells after specific gene knockdown will give insight into individual gene functions in maintaining a transformed phenotype. In this assay, RNA is extracted from antisense oligonucleotide-treated cells, and the expression of a large number of genes is determined by array analysis. Examples of phenotypic assays for cancer are summarized in Table 2.

TABLE 2

| Assay name | Target end-point | Cell line |
|---|---|---|
| Induction of | Caspase-3 activity | Breast tumor cells: MCF7 |
| apoptosis | | (p53 wt, ATCC HTB-22), T47D |
| | | (p53 mutant, ATCC HTB-133); |
| | | HMEC (normal human mammary |
| | | epithelial cells, |
| | | Clonetics); prostate tumor |
| | | cells: LNCaP (p53 wt, ATCC |
| | | CRL 1740), PC3 (p53 mutant, |
| | | ATCC CRL 1435), PrEC (normal |
| | | prostate epithelial cells, |
| | | Clonetics) |
| Cell cycle | Propidium iodide | As above |
| profile | binding of DNA/cell | |
| | cycle profile | |
| Low-density | About 200 cell | As above |
| arrays | cycle/apoptosis | |
| | genes | |
| High-density | 12,600 human genes | As above |
| arrays | | |

Inflammation

Inflammation assays center around the Th1 response that is important in several human diseases such as rheumatoid arthritis, psoriasis, multiple sclerosis and Crohn's disease. In inflammation, genes may be identified using the methods of the present invention which regulate inflammatory responses to TNF-α, IL-1-β and IFN-γ and dendritic cell/T-cell interactions (co-stimulation). These primary phenotypic assays are used to measure cytokine responsive genes (inflammation assays) using primary effector cells such as human keratinocytes, endothelial cells and fibroblasts. Functional cell responses include modulation of cell adhesion (e.g., ICAM-1 expression), inflammatory mediators (e.g., cox-2 expression), chemotaxis/angiogenesis (e.g., IL-8 expression) and extracellular matrix metabolism (e.g., MMP-9 expression). These assays identify genes that regulate responses to pro-inflammatory cytokines. The genes identified in these assays regulate various inflammatory responses.

T cells require antigen signals and co-stimulatory signals from antigen presenting cells, particularly dendritic cells, to fully respond and to differentiate into helper cells that promote intracellular (TH1) or extracellular (TH2) immunity; without co-stimulation, T cells become anergic or tolerated in vivo. This assay identifies genes in dendritic cells that, when inhibited, affect co-stimulation. These antisense oligonucleotide inhibitors have the capability of attenuating chronic, overzealous or inappropriate immune responses and of polarizing maturation of TH cells, outcomes which are therapeutically relevant to a wide range of inflammatory diseases.

In one inflammation assay, IL-2 production is measured in co-stimulation assays using dendritic cells and Jurkat T-cells. Dendritic cells are cultured in the presence of IL-4 and GM-CSF on anti-human CD3 antibody coated 96 well plates. Cells are treated with antisense oligonucleotides, and 72 hours later CTLA4-Ig fusion protein is added to block interaction of B7-1 and CD28, and to lower IL-2 secretion to baseline. Twenty-four hours later, αCD3 activated Jurkat T cells are added. Forty-eight hours later, IL-2 levels in the media are measured by ELISA. Genes that regulate co-stimulation will reduce IL-2 production. Preventing co-stimulation will decrease activation of T-cells and results in decreased inflammation. In addition, identifying genes whose inhibition results in up-regulation of co-stimulation activity of dendritic cells is also valuable.

In another inflammation assay, cells are treated with different pro-inflammatory cytokines such as IFN-γ, IL-1β and TNF-α. These pleiotropic cytokines have been implicated in the development and progression of may inflammatory, infections and autoimmune diseases. Activation of cells and tissues by these cytokines leads to the elevated expression of genes involved in cell adhesion (e.g., ICAM-1), inflammation (e.g., Cox-2), chemotaxis (e.g., Il-8) and extracellular matrix remodeling (e.g., MMP-9). These assays identify genes that, when inhibited with antisense oligonucleotide, prevent the upregulation of these proinflammatory genes. Total RNA is prepared from antisense oligonucleotide-treated cells in microtiter wells, split into 4 fractions and assayed by quantitative real-time PCR (TaqMan) in a 386 well plate for the expression of 4 different genes using, for example, the ABI Prism 7900HT detection system.

Secondary phenotypic assays include low density inflammation arrays which provides simultaneous expression analysis of many genes believed to play a role in inflammation and co-stimulation. In this assay, RNA is extracted from antisense oligonucleotide-treated cells, reverse transcribed into cDNA, PCR amplified to make a radiolabeled probe and hybridized to a glass chip with specific oligonucleotides covalently attached. Generally, about 200 genes are screened per chip.

Tertiary screens (high density arrays) may also be performed in which transcriptional profiling of cells provides insight into which genes regulate different aspects of the inflammatory process. In this assay, RNA is extracted from antisense oligonucleotide-treated cells, and the expression of a large number of genes is determined by array analysis. Phenotypic assays for inflammation are summarized in Table 3.

TABLE 3

| Assay name | Target end-point | Cell line |
|---|---|---|
| Co-stimulation | IL-2 production | Primary human |
| | | dendritic cells |
| Cytokine signaling | ICAM-1, IL-8, Cox-2, | HUVEC, |
| | MMP-9 | keratinocytes, |
| | | fibroblasts |
| Low-density DNA | 192 inflammation | Dendritic cells, |
| arrays | genes | endothelial cells, |
| | | keratinocytes, |
| | | fibroblasts |
| High-density DNA | 12,600 human genes | Dendritic cells, |
| arrays | | endothelial cells, |
| | | keratinocytes, |
| | | fibroblasts |

Metabolic Disease (Diabetes)

Resistance to insulin stimulated glucose uptake is one hallmark of non-insulin dependent diabetes. Genes which play a role in diabetes may be identified using the methods of the present invention. Antisense oligonucleotides corresponding to these genes lower blood glucose levels, regulate insulin signaling responses (adipocytes) or inhibit glucagon promotion of glucose release from glycogen (glycogenolysis/hepatocytes). Primary phenotypic assays include insulin stimulation of 2-deoxyglucose uptake in differentiated primary human adipocytes, differentiation of human adipocytes (triglyceride production/dye assay) and glucagon promotion of glucose release (glycogenolysis) in HepG2 cells. Secondary phenotypic assays include low density metabolic arrays. Tertiary phenotypic assays include high density arrays.

Adipocyte differentiation results in increased production and accumulation of fat, including triglycerides, and in secretion of the hormone leptin. The differentiation of human preadipocytes into adipocytes is determined by measuring the increase in triglycerides in cell lysates. In this assay, glycerol is liberated from the triglyceride using lipoprotein lipase. Glycerol is subsequently phosphorylated by ATP with the enzyme glycerol kinase. The glycerol-1-phosphate is oxidized to dihydroxyacetone phosphate by glycerol phosphate oxidase. Hydrogen peroxide is liberated during this reaction. Horseradish peroxidase uses the H₂O₂ liberated to oxidize 4-aminoantipyrine and 3,5 dichloro-2-hydroxybenzene sulfonate to produce a red-colored dye. The absorbance is proportional to the triglyceride concentration in the cell lysate. Leptin is measured using a commercially available assay kit.

Three tissues are responsive to insulin: fat, liver and muscle. Normal tissues respond to insulin by increased uptake of glucose. 2-deoxyglucose is also taken up in response to insulin stimulation. It is converted to 2-deoxyglucose 6-phosphate but cannot be further metabolized, and it does not efflux from the cell with the phosphate attached. ³H-2-deoxyglucose uptake in cells is used as an assay for insulin responses using, for example, Cytostar T-plates (Amersham) Antisense oligonucleotide-treated cells are serum starved overnight, and then glucose starved for an additional 30 minutes. Insulin is added to the cells, followed 15 minutes later by the addition of radiolabeled 2-deoxyglucose. The 2-deoxyglucose uptake is linear for approximately 60 minutes. During that time the radiolabel associated with cells is measured.

Glucagon stimulates both glycogenolysis and gluconeogenesis in liver cells. The net result of both glycogenolysis and gluconeogenesis is efflux of glucose from cells. The efflux of glucose from glucagon stimulated antisense oligonucleotide-treated cells is followed using a glucose assay (e.g., Molecular Probes). This assay is a one-step fluorometric assay. Glucose is detected by an enzyme-coupled reaction in which glucose reacts with glucose oxidase to form gluconolactone and H₂O₂. The H₂O₂ is detected by reacting with 10-acetyl-3,7-dihydroxyphenoxazine and horseradish peroxidase. The product formed is resorufin, which has a fluorescence emission maxima of 587 nm and is easily detected. The assay is designed to identify genes whose inhibition decreases maximal glucose effects (or insulin mimetics).

Low-density DNA arrays provide expression analysis of 190 genes believed to play a role in insulin signaling and metabolism. In this assay, RNA is extracted from antisense oligonucleotide-treated cells, reverse transcribed into cDNA, PCR amplified to make a radiolabeled probe and hybridized to a glass chip with specific oligonucleotides covalently attached.

High density (affymetrix) DNA arrays provide transcriptional profiling of adipocytes or HepG2 cells after specific gene knock-down which gives insight into individual gene functions. In this assay, RNA is extracted from antisense oligonucleotide-treated endothelial cells, and the expression of a large number of genes is determined by array analysis (U95a chip). Phenotypic assays for diabetes are summarized in Table 4.

TABLE 4

| Assay name | Target end-point | Cell line |
|---|---|---|
| Adipocyte | Triglyceride | Primary human |
| differentiation | production | adipocytes |
| 2-deoxyglucose | ³H-2-deoxyglycose | Primary human |
| uptake | uptake | adipocytes or HepG2 |
| | | (hepatoma cell line) |
| ³H-2-deoxyglucose | Glucose efflux | HepG2 |
| uptake | | |
| Low-density DNA | About 200 metabolic | Primary human |
| arrays | and diabetes genes | adipocytes or HepG2 |
| High-density DNA | 12,600 human genes | Primary human |
| arrays | | adipocytes or HepG2 |

In another embodiment, tissue fragments such as bone, liver, spleen, lung, muscle or skin are treated with a library of antisense oligonucleotides prior to incubation with a compound of interest which produces a particular response to determine whether a particular antisense oligonucleotide affects the response. If so, then the corresponding gene plays a role in the response. For example, bone fragments may be placed in culture and treated with an antisense oligonucleotide library prior to treatment with bone growth factors such as bone morphogenetic proteins (BMPs). Inhibition of bone growth by a particular antisense oligonucleotide would indicate that the target gene is involved in the response to the BMP. Similar experiments may be performed on organ cultures.

In another embodiment, the method is performed in vivo on organisms such as mice which have a particular disorder to determine which genes are involved in the disorder. For example, there are diabetic mouse models such as the db/db and ob/ob mouse in which blood glucose levels are elevated. These mice may be administered a library of antisense oligonucleotides, one oligonucleotide per mouse, followed by measurement of blood glucose levels to determine which antisense oligonucleotides result in lowered blood glucose levels. This antisense oligonucleotide then corresponds to a target gene involved in diabetes.

As used herein, the term “target nucleic acid” encompasses DNA, RNA (including pre-mRNA and mRNA) transcribed from such DNA, and also cDNA derived from such RNA. This term also includes RNAs which do not code for proteins, but which have biological effects, such as SRA (Lanz et al., Cell 97:17-27, 1999). The specific hybridization of an antisense compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation, most often inhibition, of expression of a protein encoded by the nucleic acid. In the context of the present invention, “modulation” means either an increase (stimulation) or a decrease (inhibition) in the expression of a gene. In the context of the present invention, inhibition is the preferred form of modulation of gene expression and mRNA is a preferred target.

Preferred intragenic sites for inhibition by antisense oligonucleotides include regions encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, most contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect. Antisense compounds, preferably antisense oligonucleotides, which hybridize to the target and inhibit expression of the target are identified through experimentation. The target sites to which these preferred sequences are complementary are referred to as “active sites” and are therefore preferred sites for targeting. These oligonucleotides are then referred to as “validated.”

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

For use in kits and diagnostics, antisense compounds, either alone or in combination with other antisense compounds or therapeutics, can be used as tools in differential and/or combinatorial analyses to elucidate expression patterns of a portion or the entire complement of genes expressed within cells and tissues.

Expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds preferably comprise from about 8 to about 50 nucleobases (i.e. from about 8 to about 50 linked nucleosides). Particularly preferred antisense compounds are antisense oligonucleotides, even more preferably those comprising from about 12 to about 30 nucleobases. Antisense compounds include ribozymes, external guide sequence (EGS) oligonucleotides (oligozymes), aptamers, molecular decoys and other short catalytic RNAs or catalytic oligonucleotides which hybridize to the target nucleic acid and modulate its expression.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃) —O—CH₂-[known as a methylene (methylimino) or MMI backbone], —CH₂—O—N(CH₃)—CH₂—, —CH₂—N(CH₃) —N(CH₃) —CH₂— and —O—N(CH₃) —CH₂—CH₂-[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to CO₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF3, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226. Another preferred modification is a cyclohexene nucleic acid (CeNA) in which the sugar has a double bond (PCT/IBOO/02041, herein incorporated herein by reference).

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine,

5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-oxa, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications. The terms “nucleosidic base” and “nucleobase” are further intended to include heterocyclic compounds that can serve as nucleosidic bases, including certain “universal bases” that are not nucleosidic bases in the most classical sense, but function similarly to nucleosidic bases. One representative example of such a universal base is 3-nitropyrrole.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmaco-kinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules.

Oligonucleotide-mediated modulation of expression of a target nucleic acid can be assayed in a variety of ways known in the art. For example, target RNA levels can be quantitated by, for example, Northern blot analysis, competitive PCR or reverse transcriptase PCR (RT-PCR). RNA analysis can be performed on total cellular RNA or, preferably, poly(A)+mRNA. Methods of RNA isolation are taught in, for example, Ausubel et al. (Short Protocols in Molecular Biology, 2^{nd} Ed., pp. 4-1 to 4-13, Greene Publishing and John Wiley & Sons, New York, 1992).

Alternatively, total RNA can be prepared from cultured cells or tissue using the RNeasy-96 kit (QIAGEN, Inc., Valencia, Calif.) for the high throughput preparation of RNA carried out according to the manufacturer's instructions. Optionally, a DNase step is included to remove residual DNA prior to RT-PCR. To improve efficiency and accuracy, the repetitive pipeting steps and elution step have been automated using a QIAGEN Bio-Robot 9604. Essentially after lysing of the oligonucleotide treated cell cultures in situ, the plate is transferred to the robot deck where the pipeting, DNase treatment and elution steps are carried out. RT-PCR can be conveniently accomplished using, for example, the ABI PRISM 7700 sequence detection system (PE-Applied Biosystems, Foster City, Calif.) according to the manufacturer's instructions. Other methods of PCR are also known in the art.

Target protein levels can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a protein encoded by a target nucleic acid can be identified and obtained from a variety of sources, such as the MSRS catalog of antibodies (Aerie Corporation, Birmingham, Mich.), or can be prepared via conventional antibody generation methods. Methods for preparation of polyclonal, monospecific (“antipeptide”) and monoclonal antisera are taught by, for example, Ausubel et al. (supra., pp. 11-3 to 11-54).

Because it is preferred to assay the antisense oligonucleotide library parallel to the automated synthesis process described above, preferred means of assaying are suitable for use in multi-well (e.g., 96- or 384-well) plates and with robotic means. Accordingly, automated RT-PCR is preferred for assaying target nucleic acid levels, and automated ELISA is preferred for assaying target protein levels.

When RT-PCR is used to evaluate the activities of the compounds, cells are plated into multi-well plates (typically, 96-well) and treated with the antisense oligonucleotide library or control oligonucleotide (one oligonucleotide per well). Cells are harvested and lysed, and the lysates are introduced into an apparatus where RT-PCR is carried out. A raw data file is generated, and the data is downloaded and compiled. Spreadsheet files with data charts are generated, and the experimental data is analyzed. Data from the assays on each oligonucleotide ate compiled and statistical parameters are automatically determined.

In preferred embodiments, an activity profile is prepared for each screened compound. Compounds that fail to meet threshold values for activity are then removed, and the remaining compounds are referred to as “prevalidated” oligonucleotides.

The antisense oligonucleotides screened using the methods of the present invention may be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds screened using the methods of the present invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

In one embodiment of the present invention, an animal suspected of having a disease or disorder is administered a library of antisense compounds in accordance with this invention. The compounds of the invention can be utilized in pharmaceutical compositions by adding an effective amount of an antisense compound to a suitable pharmaceutically acceptable diluent or carrier.

The present invention also includes pharmaceutical compositions and formulations which include individual members of the library of antisense compounds of the invention. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include polyamino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999) each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The antisense compounds may be prepared and formulated as emulsions. Emulsions are typically heterogeneous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S. T. P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9^{th} Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18^{th} Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15^{th} Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15^{th} Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same.

### EXAMPLES

### Example 1

Nucleoside Phosphoramidites for Oligonucleotide Synthesis Deoxy and 2′-alkoxy Amidites

2′-Deoxy and 2′-methoxy beta-cyanoethyldiisopropyl phosphoramidites were purchased from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, the standard cycle for unmodified oligonucleotides was utilized, except the wait step after pulse delivery of tetrazole and base was increased to 360 seconds.

Oligonucleotides containing 5-methyl-2′-deoxycytidine (5-Me-C) nucleotides were synthesized according to published methods [Sanghvi, et. al., Nucleic Acids Research, 1993, 21, 3197-3203] using commercially available phosphoramidites (Glen Research, Sterling Va. or ChemGenes, Needham Mass.).

2′-Fluoro Amidites

2′-Fluorodeoxyadenosine Amidites

2′-fluoro oligonucleotides were synthesized as described previously [Kawasaki, et. al., J. Med. Chem., 1993, 36, 831-841] and U.S. Pat. No. 5,670,633, herein incorporated by reference. Briefly, the protected nucleoside N6-benzoyl-2′-deoxy-2′-fluoroadenosine was synthesized utilizing commercially available 9-beta-D-arabinofuranosyladenine as starting material and by modifying literature procedures whereby the 2′-alpha-fluoro atom is introduced by a SN²-displacement of a 2′-beta-trityl group. Thus N6-benzoyl-9-beta-D-arabinofuranosyladenine was selectively protected in moderate yield as the 3′,5′-ditetrahydropyranyl (THP) intermediate. Deprotection of the THP and N6-benzoyl groups was accomplished using standard methodologies and standard methods were used to obtain the 5′-dimethoxytrityl-(DMT) and 5′-DMT-3′-phosphoramidite intermediates.

2′-Fluorodeoxyguanosine

The synthesis of 2′-deoxy-2′-fluoroguanosine was accomplished using tetraisopropyldisiloxanyl (TPDS) protected 9-beta-D-arabinofuranosylguanine as starting material, and conversion to the intermediate diisobutyryl-arabinofuranosylguanosine. Deprotection of the TPDS group was followed by protection of the hydroxyl group with THP to give diisobutyryl di-THP protected arabinofuranosylguanine. Selective O-deacylation and triflation was followed by treatment of the crude product with fluoride, then deprotection of the THP groups. Standard methodologies were used to obtain the 5′-DMT- and 5′-DMT-3′-phosphoramidites.

2′-Fluorouridine

Synthesis of 2′-deoxy-2′-fluorouridine was accomplished by the modification of a literature procedure in which 2,2′-anhydro-1-beta-D-arabinofuranosyluracil was treated with 70% hydrogen fluoride-pyridine. Standard procedures were used to obtain the 5′-DMT and 5′-DMT-3′phosphoramidites.

2′-Fluorodeoxycytidine

2′-deoxy-2′-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′phosphoramidites.

2′-O-(2-Methoxyethyl) Modified Amidites

2′-O-Methoxyethyl-substituted nucleoside amidites are prepared as follows, or alternatively, as per the methods of Martin, P., Helvetica Chimica Acta, 1995, 78, 486-504.

2,2′-Anhydro[1-(beta-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 h) to give a solid that was crushed to a light tan powder (57 g, 85% crude yield). The NMR spectrum was consistent with the structure, contaminated with phenol as its sodium salt (ca. 5%). The material was used as is for further reactions (or it can be purified further by column chromatography using a gradient of methanol in ethyl acetate (10-25%) to give a white solid, mp 222-4° C.).

2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product. Additional material was obtained by reworking impure fractions.

2′-O-Methoxyethyl-5′-O-dimethoxytrityl-5-methyluridine

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL). The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/hexane/acetone (5:5:1) containing 0.5% Et₃NH. The pure fractions were evaporated to give 164 g of product. Approximately 20 g additional was obtained from the impure fractions to give a total yield of 183 g (57%).

3′-O-Acetyl-2′-O-methoxyethyl-5¹-O-dimethoxytrityl-5-methyluridine

2′1-O-Methoxyethyl-5′1O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by TLC by first quenching the TLC sample with the addition of MeOH. Upon completion of the reaction, as judged by TLC, MeOH (50 mL) was added and the mixture evaporated at 35° C. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/hexane(4:1). Pure product fractions were evaporated to yield 96 g (84%). An additional 1.5 g was recovered from later fractions.

3′-O-Acetyl-2′-O-methoxyethyl-5′-O-dimethoxytrityl-5-methyl-4-triazoleuridine

A first solution was prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5° C. and stirred for 0.5 h using an overhead stirrer. POCl₃ was added dropwise, over a 30 minute period, to the stirred solution maintained at 0-10° C., and the resulting mixture stirred for an additional 2 hours. The first solution was added dropwise, over a 45 minute period, to the latter solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with lx300 mL of NaHCO₃ and 2×300 mL of saturated NaCl, dried over sodium sulfate and evaporated. The residue was triturated with EtOAc to give the title compound.

2′-O-Methoxyethyl-5′-O-dimethoxytrityl-5-methylcytidine

A solution of 3′1—acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100° C. for 2 hours (TLC showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

N4-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, TLC showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/hexane (1:1) containing 0.5% Et₃NH as the eluting solvent. The pure product fractions were evaporated to give 90 g (90%) of the title compound.

N4-Benzoyl-2′-O-methoxyethyl-5′-O-dimethoxytrityl-5-methylcytidine-3′-amidite

N4-Benzoyl-2′-O-methoxyethyl-5′-O-dimethoxytrityl-5-methylcytidine (74 g, 0.10 M) was dissolved in CH₂Cl₂ (1 L). Tetrazole diisopropylamine (7.1 g) and 2-cyanoethoxy-tetra-(isopropyl)phosphite (40.5 mL, 0.123 M) were added with stirring, under a nitrogen atmosphere. The resulting mixture was stirred for 20 hours at room temperature (TLC showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄ and concentrated. The residue obtained was chromatographed on a 1.5 kg silica column using EtOAc/hexane (3:1) as the eluting solvent. The pure fractions were combined to give 90.6 g (87%) of the title compound. 2′-O-(Aminooxyethyl) nucleoside amidites and 2′-O-(dimethylaminooxyethyl) Nucleoside Amidites

2′-(Dimethylaminooxyethoxy) Nucleoside Amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine. 5′-O-tert-Butyldiphenylsilyl-0²-2′-anhydro-5-methyluridine

O²-2′-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, l.leq, 0.458 mmol) was added in one portion. The reaction was stirred for 16 h at ambient temperature. TLC (Rf 0.22, ethyl acetate) indicated a complete reaction. The solution was concentrated under reduced pressure to a thick oil. This was partitioned between dichloromethane (1 L) and saturated sodium bicarbonate (2xl L) and brine (1 L). The organic layer was dried over sodium sulfate and concentrated under reduced pressure to a thick oil. The oil was dissolved in a 1:1 mixture of ethyl acetate and ethyl ether (600 mL) and the solution was cooled to −10° C. The resulting crystalline product was collected by filtration, washed with ethyl ether (3×200 mL) and dried (40° C., 1 mm Hg, 24 h) to 149 g (74.8%) of white solid. TLC and NMR were consistent with pure product.

5′1-O-tert-Butyldiphenylsilyl-2′-O-(2-hydroxyethyl)-5-methyluridine

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-Butyldiphenylsilyl-O ²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 1600 C was reached and then maintained for 16 h (pressure <100 psig). The reaction vessel was cooled to ambient and opened. TLC (Rf 0.67 for desired product and Rf 0.82 for ara-T side product, ethyl acetate) indicated about 70% conversion to the product. In order to avoid additional side product formation, the reaction was stopped, concentrated under reduced pressure (10 to 1 mm Hg) in a warm water bath (40-100° C.) with the more extreme conditions used to remove the ethylene glycol. [Alternatively, once the low boiling solvent is gone, the remaining solution can be partitioned between ethyl acetate and water. The product will be in the organic phase.] The residue was purified by column chromatography (2 kg silica gel, ethyl acetate-hexanes gradient 1:1 to 4:1). The appropriate fractions were combined, stripped and dried to product as a white crisp foam (84 g, 50%), contaminated starting material (17.4 g) and pure reusable starting material 20 g. The yield based on starting material less pure recovered starting material was 58%. TLC and NMR were consistent with 99% pure product.

2′1-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol). It was then dried over P₂O₅ under high vacuum for two days at 40° C. The reaction mixture was flushed with argon and dry THF (369.8 mL, Aldrich, sure seal bottle) was added to get a clear solution. Diethyl-azodicarboxylate (6.98 mL, 44.36 mmol) was added dropwise to the reaction mixture. The rate of addition is maintained such that resulting deep red coloration is just discharged before adding the next drop. After the addition was complete, the reaction was stirred for 4 hrs. By that time TLC showed the completion of the reaction (ethylacetate:hexane, 60:40). The solvent was evaporated in vacuum. Residue obtained was placed on a flash column and eluted with ethyl acetate:hexane (60:40), to get 2′-O-([2-phthalimidoxy)ethyl]-5′-t-butyldiphenylsilyl-5-methyluridine as white foam (21.819 g, 86%).

5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate was washed with ice cold CH₂Cl₂ and the combined organic phase was washed with water, brine and dried over anhydrous Na₂SO₄. The solution was concentrated to get 2′-O-(aminooxyethyl) thymidine, which was then dissolved in MeOH (67.5 mL). To this formaldehyde (20% aqueous solution, w/w, 1.1 eq.) was added and the resulting mixture was stirred for 1 h. Solvent was removed under vacuum; residue chromatographed to get 5′-O-tert-butyldiphenylsilyl-2′-O-[(2-formadoximinooxy) ethyl]-5-methyluridine as white foam (1.95 g, 78%).

5′-O-tert-Butyldiphenylsilyl-2′-O-[N,N-dimethylaminooxyethyl]-5-methyluridine

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL). Sodium cyanoborohydride (0.39 g, 6.13 mmol) was added to this solution at 10° C. under inert atmosphere. The reaction mixture was stirred for 10 minutes at 10° C. After that the reaction vessel was removed from the ice bath and stirred at room temperature for 2 h, the reaction monitored by TLC (5% MeOH in CH₂Cl₂). Aqueous NaHCO₃ solution (5%, 10 mL) was added and extracted with ethyl acetate (2×20 mL). Ethyl acetate phase was dried over anhydrous Na₂SO₄, evaporated to dryness. Residue was dissolved in a solution of 1M PPTS in MeOH (30.6 mL). Formaldehyde (20% w/w, 30 mL, 3.37 mmol) was added and the reaction mixture was stirred at room temperature for 10 minutes. Reaction mixture cooled to 10° C. in an ice bath, sodium cyanoborohydride (0.39 g, 6.13 mmol) was added and reaction mixture stirred at 10° C. for 10 minutes. After 10 minutes, the reaction mixture was removed from the ice bath and stirred at room temperature for 2 hrs. To the reaction mixture 5% NaHCO₃ (25 mL) solution was added and extracted with ethyl acetate (2×25 mL). Ethyl acetate layer was dried over anhydrous Na₂SO₄ and evaporated to dryness. The residue obtained was purified by flash column chromatography and eluted with 5% MeOH in CH₂Cl₂ to get 5′-O-tert-butyldiphenylsilyl-2′-O-[N,N-dimethylaminooxyethyl]-5-methyluridine as a white foam (14.6 g, 80%).

2′1-O-(dimethylaminooxyethyl)-5-methyluridine

Triethylamine trihydrofluoride (3.91 mL, 24.0 mmol) was dissolved in dry THF and triethylamine (1.67 mL, 12 mmol, dry, kept over KOH). This mixture of triethylamine-2HF was then added to 5′-O-tert-butyldiphenylsilyl-2′-O-[N,N-dimethylaminooxyethyl]-5-methyluridine (1.40 g, 2.4 mmol) and stirred at room temperature for 24 hrs. Reaction was monitored by TLC (5% MeOH in CH₂Cl₂). Solvent was removed under vacuum and the residue placed on a flash column and eluted with 10% MeOH in CH₂Cl₂ to get 2′-O-(dimethylaminooxyethyl)-5-methyluridine (766 mg, 92.5%).

5′-O-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C. It was then co-evaporated with anhydrous pyridine (20 mL). The residue obtained was dissolved in pyridine (llmL) under argon atmosphere. 4-dimethylaminopyridine (26.5 mg, 2.60 mmol), 4,4′-dimethoxytrityl chloride (880 mg, 2.60 mmol) was added to the mixture and the reaction mixture was stirred at room temperature until all of the starting material disappeared. Pyridine was removed under vacuum and the residue chromatographed and eluted with 10% MeOH in CH₂Cl₂ (containing a few drops of pyridine) to get 5′-O-DMT-2′-O-(dimethylamino-oxyethyl)-5-methyluridine (1.13 g, 80%).

5′-O-DMT-2′-O-(2-N,N-dimethylaminooxyethyl)-5-methyluridine-3′-[(2-cyanoethyl)-N,N-diisopropylphosphoramidite]

5′—DMT-2′-O-(dimethylaminooxyethyl)-5-methyluridine (1.08 g, 1.67 mmol) was co-evaporated with toluene (20 mL). To the residue N,N-diisopropylamine tetrazonide (0.29 g, 1.67 mmol) was added and dried over P₂O₅ under high vacuum overnight at 40° C. Then the reaction mixture was dissolved in anhydrous acetonitrile (8.4 mL) and 2-cyanoethyl-N,N,N¹,N¹-tetraisopropylphosphoramidite (2.12 mL, 6.08 mmol) was added. The reaction mixture was stirred at ambient temperature for 4 hrs under inert atmosphere. The progress of the reaction was monitored by TLC (hexane:ethyl acetate 1:1). The solvent was evaporated, then the residue was dissolved in ethyl acetate (70 mL) and washed with 5% aqueous NaHCO₃ (40 mL). Ethyl acetate layer was dried over anhydrous Na₂SO₄ and concentrated. Residue obtained was chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%).

2′-(Aminooxyethoxy) Nucleoside Amidites

2′-(Aminooxyethoxy) nucleoside amidites [also known in the art as 2′-O-(aminooxyethyl) nucleoside amidites] are prepared as described in the following paragraphs. Adenosine, cytidine and thymidine nucleoside amidites are prepared similarly.

N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with aminor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-0-(4,4′-dimethoxytrityl)guanosine which may be reduced to provide 2-N-isobutyryl-6-O-diphenylcarbamoyl-2′-O-(2-hydroxyethyl)-5′-O-(4,4′-dimethoxytrityl)guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-([2-phthalmidoxy]ethyl)-5′-O-(4,4′-dimethoxytrityl)guanosine-3′-[(2-cyanoethyl)-N,N-diisopropylphosphoramidite].

2′-dimethylaminoethoxyethoxy (2′-DMAEOE) Nucleoside Amidites

2′-dimethylaminoethoxyethoxy nucleoside amidites (also known in the art as 2′-O-dimethylaminoethoxyethyl, i.e., 2′-O—CH₂—O—CH₂—N(CH₂)₂, or 2′-DMAEOE nucleoside amidites) are prepared as follows. Other nucleoside amidites are prepared similarly.

2′-O-[2(2-N,N-dimethylaminoethoxy)ethyl]-5-methyl Uridine

2[2-(Dimethylamino)ethoxy]ethanol (Aldrich, 6.66 g, 50 mmol) is slowly added to a solution of borane in tetrahydrofuran (1 M, 10 mL, 10 mmol) with stirring in a 100 mL bomb. Hydrogen gas evolves as the solid dissolves. O²-,2′-anhydro-5-methyluridine (1.2 g, 5 mmol), and sodium bicarbonate (2.5 mg) are added and the bomb is sealed, placed in an oil bath and heated to 155° C. for 26 hours. The bomb is cooled to room temperature and opened. The crude solution is concentrated and the residue partitioned between water (200 mL) and hexanes (200 mL). The excess phenol is extracted into the hexane layer. The aqueous layer is extracted with ethyl acetate (3×200 mL) and the combined organic layers are washed once with water, dried over anhydrous sodium sulfate and concentrated. The residue is columned on silica gel using methanol/methylene chloride 1:20 (which has 2% triethylamine) as the eluent. As the column fractions are concentrated a colorless solid forms which is collected to give the title compound as a white solid.

5′-O-dimethoxytrityl-2′-O-[2(2-N,N-dimethylaminoethoxy)ethyl)]-5-methyl Uridine

To 0.5 g (1.3 mmol) of 2′-O-[2(2-N,N-dimethylaminoethoxy)ethyl)]-5-methyl uridine in anhydrous pyridine (8 mL), triethylamine (0.36 mL) and dimethoxytrityl chloride (DMT-Cl, 0.87 g, 2 eq.) are added and stirred for 1 hour. The reaction mixture is poured into water (200 mL) and extracted with CH₂Cl₂ (2×200 mL). The combined CH₂Cl₂ layers are washed with saturated NaHCO₃ solution, followed by saturated NaCl solution and dried over anhydrous sodium sulfate. Evaporation of the solvent followed by silica gel chromatography using MeOH:CH₂Cl₂:Et₃N (20:1, v/v, with 1% triethylamine) gives the title compound.

5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) are added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture is stirred overnight and the solvent evaporated. The resulting residue is purified by silica gel flash column chromatography with ethyl acetate as the eluent to give the title compound.

### Example 2

Oligonucleotide Synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle was replaced by 0.2 M solution of 3H-1,2-benzodithiole-3-one 1,1-dioxide in acetonitrile for the stepwise thiation of the phosphite linkages. The thiation wait step was increased to 68 sec and was followed by the capping step. After cleavage from the CPG column and deblocking in concentrated ammonium hydroxide at 55° C. (18 h), the oligonucleotides were purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, and 5,719,262, herein incorporated by reference.

### Example 5

Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

[2′-O-Me]—[2′-deoxy]—[2′-O-Me]Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 s repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample was again lyophilized to dryness. The pellet is resuspended in 1M TBAF in THF for 24 hrs at room temperature to deprotect the 2′ positions. The reaction is then quenched with 1M TEAA and the sample is then reduced to ½ volume by rotovac before being desalted on a G25 size exclusion column. The oligo recovered is then analyzed spectrophotometrically for yield and for purity by capillary electrophoresis and by mass spectrometry.

[2′-O-(2-Methoxyethyl)]—[2′-deoxy]—[2′-O-(Methoxyethyl)] Chimeric Phosphorothioate Oligonucleotides

[2′-O-(2-methoxyethyl)]—[2′-deoxy]—[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

[2′-O— (2-Methoxyethyl)Phosphodiester]—[2′-deoxy Phosphorothioate]—[2′-O-(2-Methoxyethyl) Phosphodiester] Chimeric Oligonucleotides

[2′-O-(2-methoxyethyl phosphodiester]—[2′-deoxy phosphorothioate]—[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 6

Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Oligonucleotide Synthesis -96 Well Plate Format

Oligonucleotides were synthesized via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites were purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

Oligonucleotide Analysis—96 Well Plate Format

The concentration of oligonucleotide in each well was assessed by dilution of samples and UV absorption spectroscopy. The full-length integrity of the individual products was evaluated by capillary electrophoresis (CE) in either the 96 well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

Cell culture and oligonucleotide treatment

The effect of antisense compounds on target nucleic acid expression can be tested in any of a variety of cell types provided that the target nucleic acid is present at measurable levels. This can be routinely determined using, for example, PCR or Northern blot analysis. The following 5 cell types are provided for illustrative purposes, but other cell types can be routinely used, provided that the target is expressed in the cell type chosen. This can be readily determined by methods routine in the art, for example Northern blot analysis, Ribonuclease protection assays, or RT-PCR. T-24 cells:

The human transitional cell bladder carcinoma cell line T-24 is obtained from the American Type Culture Collection (ATCC) (Manassas, Va.). T-24 cells are routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells are routinely passaged by trypsinization and dilution when they reach 90% confluence.

A549 Cells:

The human lung carcinoma cell line A549 is obtained from the American Type Culture Collection (ATCC) (Manassas, Va.). A549 cells are routinely cultured in DMEM basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells are routinely passaged by trypsinization and dilution when they reach 90% confluence.

NHDF Cells:

Human neonatal dermal fibroblast (NHDF) are obtained from the Clonetics Corporation (Walkersville Md.). NHDFs are routinely maintained in Fibroblast Growth Medium (Clonetics Corporation, Walkersville Md.) supplemented as recommended by the supplier. Cells are maintained for up to 10 passages as recommended by the supplier.

HEK Cells:

Human embryonic keratinocytes (HEK) are obtained from Clonetics. HEKs are routinely maintained in Keratinocyte Growth Medium (Clonetics Corporation, Walkersville Md.) formulated as recommended by the supplier. Cells are routinely maintained for up to 10 passages as recommended by the supplier.

HUVEC:

Human umbilical vein endothelial cells (HUVECs) are obtained from Clonectics. HUVECs are routinely maintained in the designated EBM medium supplemented with 10% fetal bovine serum (FBS). Cells are used from passages two to ten at 80-90% confluency.

Dendritic Cells:

Primary human dendritic cells are obtained from clonetics and cultured in serum-free lymphocyte growth medium (LGM-3, Clonetics) in the presence of 500 U/mL each of granulocyte macrophage colony stimulating factor (GM-CSF) and interleukin-4 (IL-4). Fresh medium containing the cytokines is replaced every 48 hours.

Treatment with Antisense Compounds:

When cells reach 80% confluency, they are treated with an oligonucleotide library. For cells grown in 96-well plates, wells are washed once with 200 μL OPTI-MEM™-L reduced-serum medium (Gibco BRL) and then treated with 130 μL of OPTI-MEM™-1 containing 3.75 μg/mL LIPOFECTIN™ (Gibco BRL) and each well is incubated with the desired concentration of a different antisense oligonucleotide. After 4-7 hours of treatment, the medium is replaced with fresh medium and cells are treated with the desired test compound. Cells are harvested 16-24 hours after oligonucleotide treatment.

### Example 10

Dendritic Cell Co-Stimulation Assay Antisense Library Screen

Cell Culture and Reagents—A primary dendritic cell (DC)-based T cell co-stimulation assay measuring IL-2 production was used to identify active antisense oligonucleotide from a library of 240 antisense oligonucleotides. Primary human dendritic cells (Clonetics) were cultured in serum-free lymphocyte growth medium (LGM-3, Clonetics) in the presence of 500 U/ml each of recombinant GM-CSF and IL-4 (R&D Systems, Minneapolis, Minn.). Fresh medium containing the cytokines was replaced every 48 hours. D1.1 and Jurkat T cells were cultured in RPMI 1640 medium supplemented with 10% heat-inactivated fetal bovine serum (Sigma, St. Louis, Mo.), 10 mM HEPES, pH 7.2, 50 μM 2-mercaptoethanol, 2 mM L-glutamine, 100 U/ml penicillin and 100 μg/ml streptomycin (Gibco, Grand Island, N.Y.). E. coli 0128:B12 lipopolysaccharide (LPS) was from Difco Laboratories.

Library Development—An antisense oligonucleotide library (229 oligonucleotides, each corresponding to a different target gene) was applied to cells (one per well). Active sequences were identified by real-time RT-PCR screening of cell lines in 96-well plate format or by Northern blotting, synthesized in 1 micromole scale and aliquoted into 96-well plates at 3 micromolar concentrations using robotics.

Dendritic cells (DCs) were plated at 6500 cells/well on anti-CD3 (UCHT1, Pharmingen-BD, San Diego, Calif.) coated plates in 500 U/mL GM-CSF and IL-4 for 24 hours. DCs were then transfected with 200 nM of each oligonucleotide with Lipofectin™ reagent (Gibco, Grand Island, N.Y.) using standard protocols. Briefly, oligonucleotides were pre-mixed with LIPOFECTIN™ in OptiMEM serum-free medium (Gibco) at 3 μL per 100 nM oligonucleotide per mL and incubated with DCs for 4 hours at 37° C. Following transfection, LGM-3 plus cytokines was replaced and DCs were cultured for 48 hours before co-culture of DCs with Jurkat T cells in complete RPMI medium as described above. Culture supernatants were collected 48 hours later and assayed for IL-2 content by sandwich ELISA (IL-2 DuoSet, R & D systems, Minneapolis, Minn.).

The results are shown in Table 5. The antisense oligonucleotides from the library which were the most effective at inhibiting IL-2 production and inhibited IL-2 production in a dose-dependent manner were targeted to genes 54, 86, 212, 207 and 104. This shows that these genes play a role in T cell-mediated stimulation of IL-2 production by dendritic cells.

TABLE 5

| | inhibition of IL-2 |
|---|---|
| Oligonucleotide | production |
| 23 | +++ |
| 43 | ++ |
| 53 | ++++ |
| 54 | +++ |
| 70 | ++ |
| 75 | ++ |
| 37 | ++ |
| 44 | ++ |
| 34 | +++ |
| 140 | ++++ |
| 105 | ++ |
| 104 | +++ |
| 133 | +++ |
| 139 | +++ |
| 106 | +++ |
| 135 | +++ |
| 113 | +++ |
| 86 | ++ |
| 33 | ++ |
| 198 | +++ |
| 209 | +++ |
| 174 | ++ |
| 158 | ++ |
| 207 | ++ |
| 212 | ++ |
| 197 | ++ |
| 159 | ++ |
| 201 | ++ |

### Example 11

Keratinocyte Cell (KC) Activation Assay

Neonatal KC and KGM-2 media were purchased from Clonetics (Palo Alto, Calif.). KC were seeded in 96 well plates the day before at 20,000 cells/cm2. KC were treated with 200 nM antisense oligonucleotides (one oligonucleotide per well) in 6 μg/ml Lipofectin/Optimem for 4 hours. After antisense treatment, fresh media was added and the cells were incubated for 48 hours. The cells were then induced overnight with fresh media containing 10 ng/ml human TNF-α. The supernatant was assayed for IL-8 production using either the R&D Systems or BioSource ELISA reagents. The KC adherent to the 96 well plates were fixed with 2% formaldehyde for 15 minutes at room temperature and then assayed for ICAM-1 expression using the 84H10 antibody (Immunotech). IL-8 and ICAM-1 expression was normalized to endogenous biotin levels using a streptavidin-beta galactosidase conjugate (Roche Molecular Biochemicals) The most active oligonucleotides were 158 (70.7% of control for ICAM-1, 32.9% of control for IL-8), 159 (59.3% of control for ICAM-1, 60.8% of control for IL-8), 168 (63.9% of control for ICAM-1, 60.5% of control for IL-8), 169 (68.3% of control for ICAM-1, 57.5% of control for IL-8), 201 (51.3% of control for ICAM-1, 37.9% of control for ICAM-1), 205 (58% of control for ICAM-1 and 75.1% of control for IL-8) and 220 (64.8% of control for ICAM-1 and 58% of control for IL-8). These oligonucleotides correspond to gene products involved in the response of keratinocytes to TNF-α.

### Example 12

Screening of Antisense Oligonucleotide Libraries in Diabetic Mouse Model

Db/db mice are used as a model of Type II diabetes. These mice are hyperglycemic, obese, hyperlipidemic and insulin resistant. The db/db phenotype is due to a mutation in the leptin receptor on a C57BLKS background. However, a mutation in the leptin gene can produce obesity without diabetes (ob/ob mice). Leptin is a hormone produced by adipocytes that regulates appetite. Animals or humans with leptin deficiencies become obese. Heterozygous db/wt mice (known as lean littermates) do not display the hyperglycemia/hyperlipidemia or obesity phenotype and are used as controls.

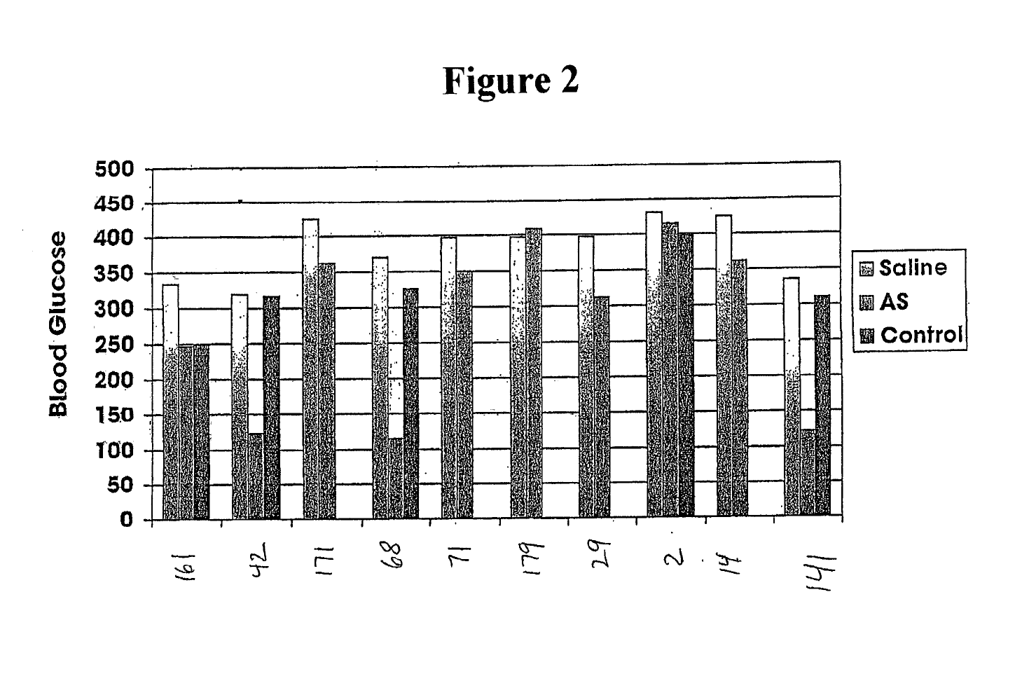

This model was used to screen antisense oligonucleotide libraries in vivo to determine genes which play a role in the production or maintenance of elevated glucose levels. Male db/db mice and lean (heterozygous, i.e., db/wt) littermates (age 9 weeks at time 0) were divided into matched groups with the same average blood glucose levels and treated by intraperitoneal injection once a week with saline or one of the following antisense oligonucleotides at a dose of 50 mg/kg: 161, 42, 171, 68, 71, 179, 29, 2, 14 and 141. Treatment was continued for 4 weeks with blood glucose levels being measured on day 0, 7, 14, 21 and 28. The results are shown in FIG. 2. Antisense oligonucleotides which significantly lowered blood glucose levels (42, 68 and 141) correspond to genes involved in Type 2 diabetes.

### Example 13

Endothelial cell tube formation assay

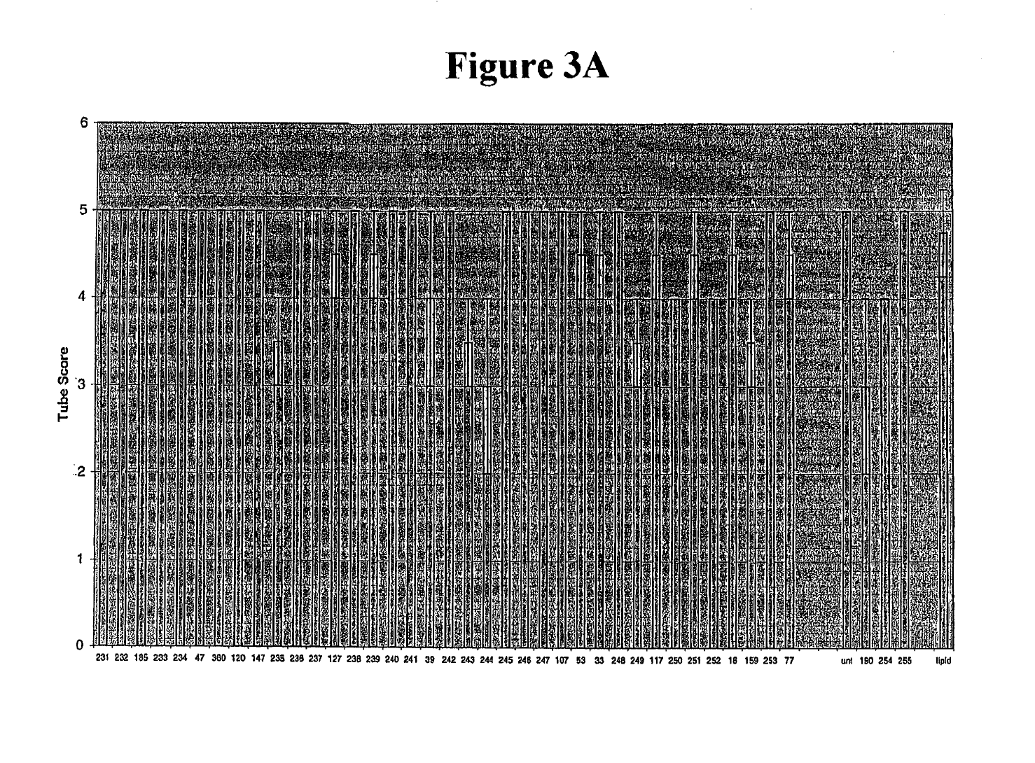

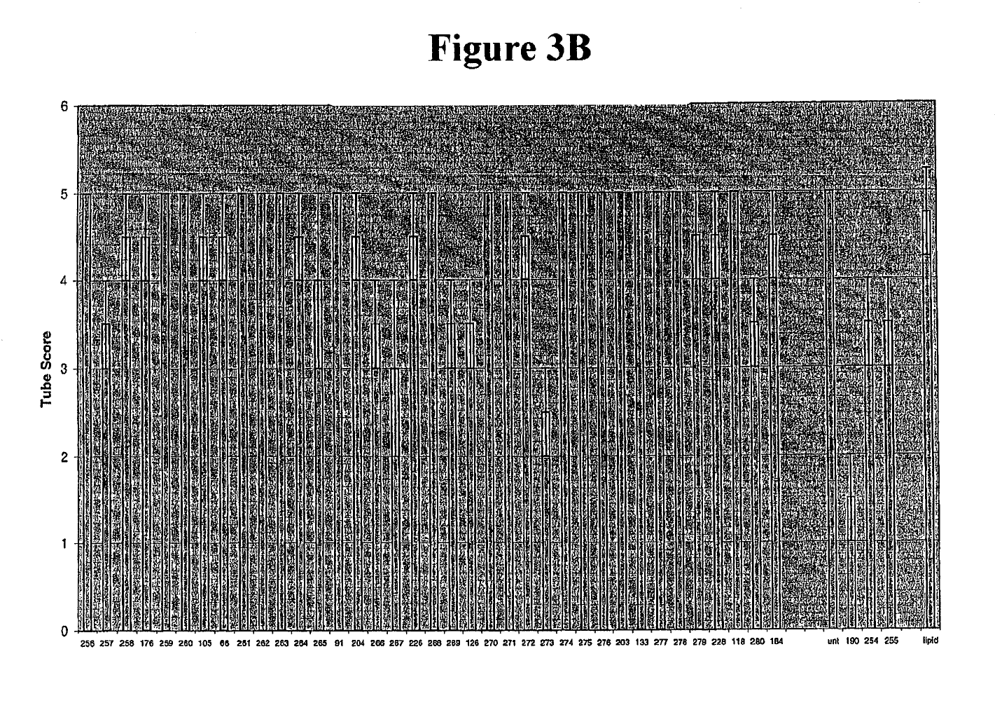

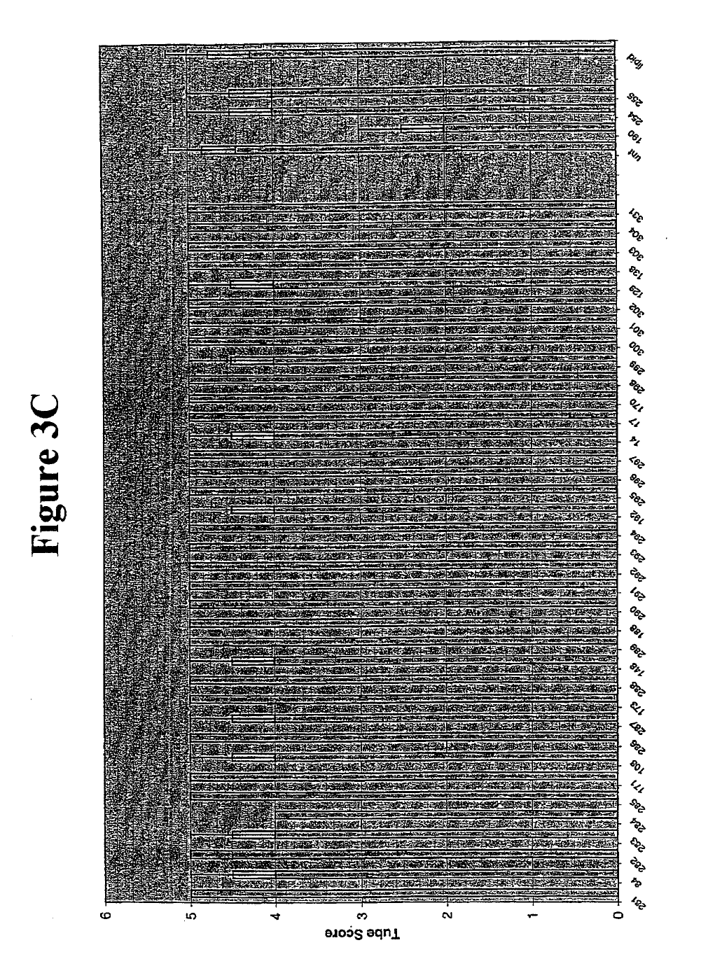

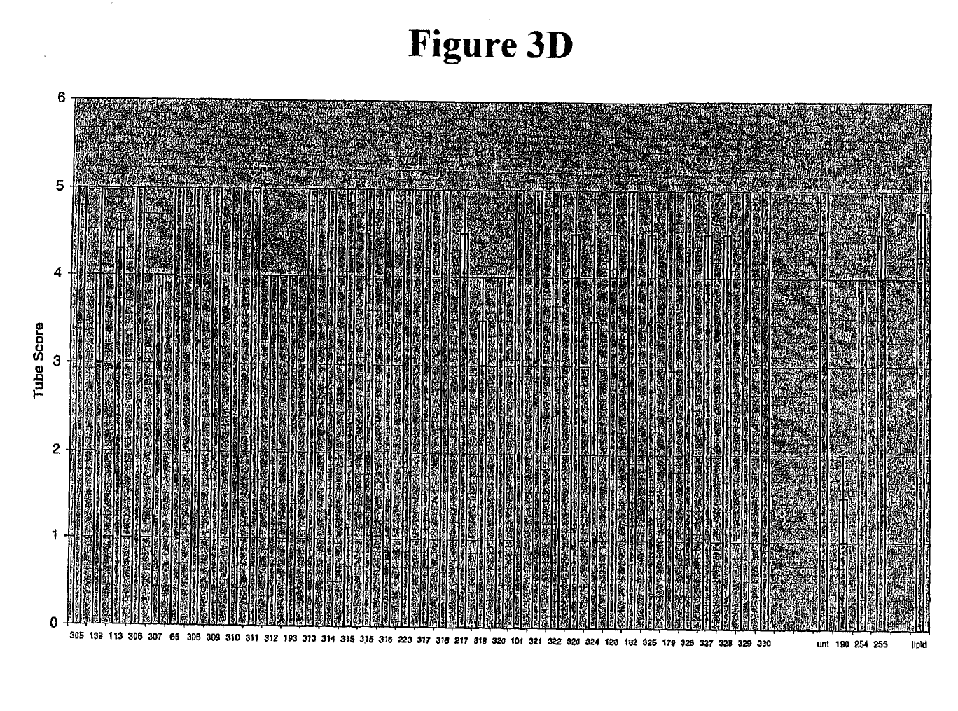

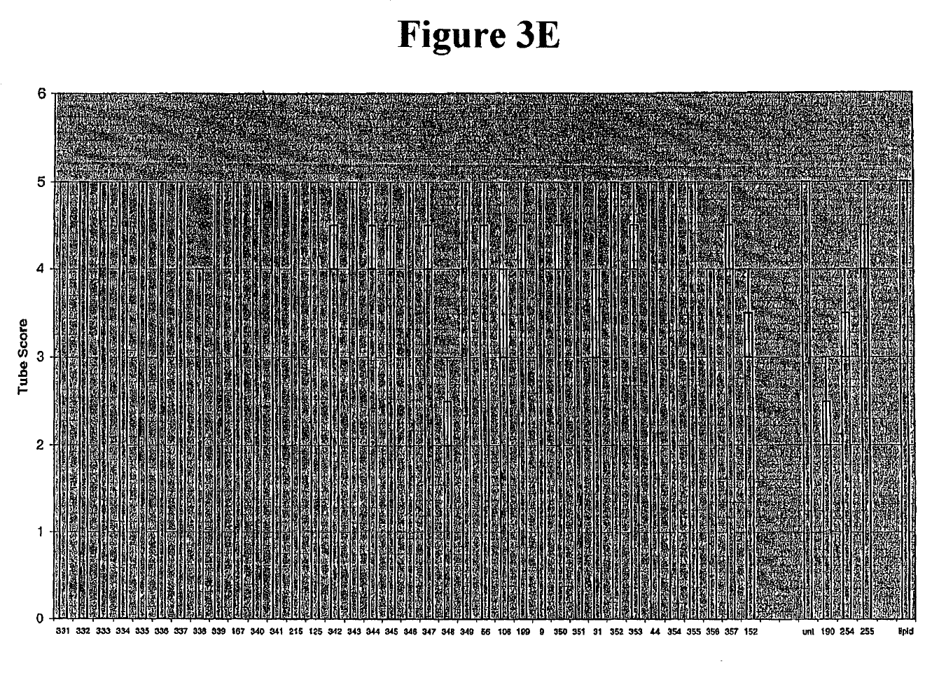

This assay was performed using the in vitro angiogenesis assay kit (Chemicon International, Temecula, Calif.). Briefly, an antisense oligonucleotide library (185 oligonucleotides) was used to transfect human umbilical vein endothelial cells (HUVEC) with LIPOFECTIN™ in 96-well plates (one antisense oligonucleotide per well, 100 μL final media volume). Forty-six hours post-transfection, plates were prepared using a mix that was 67.5% extracellular matrix (ECM), 22.5% phosphate buffered saline (PBS) and 10% 10×ECM dilution buffer (Chemicon). Forty μL of the mixture was added to each well and the plates were incubated at 37° C. for at least one hour to let the matrix solidify. Forty-eight hours post-transfection, cells were removed with trypsin and transferred to ECM-coated plates in regular growth media. Cells were incubated for 16-20 hours, then visually inspected under the microscope. Each well was assigned a score between 1 and 6 depending on the extent of tube formation. A score of 1 refers to a well with no tube formation, indicating complete inhibition by the oligonucleotide, while a score of 6 is given to wells with 100% of cells forming tubes, indicating no inhibition by the oligonucleotide. Intermediate scores indicate different levels of inhibition. The results are summarized in FIGS. 3A-3E. Unt=untreated, 254=positive control, 190=positive control, 255=negative control, lipid=negative control. Antisense oligonucleotides which significantly decreased endothelial tube formation include 190, 273, 348, 152, 243, 235, 249, 244 and 159. Thus, these oligonucleotides correspond to genes involved in promoting angiogenesis and are potential angiogenesis inhibitors.

### Example 14

Matrix Metalloprotease (MMP) Assay

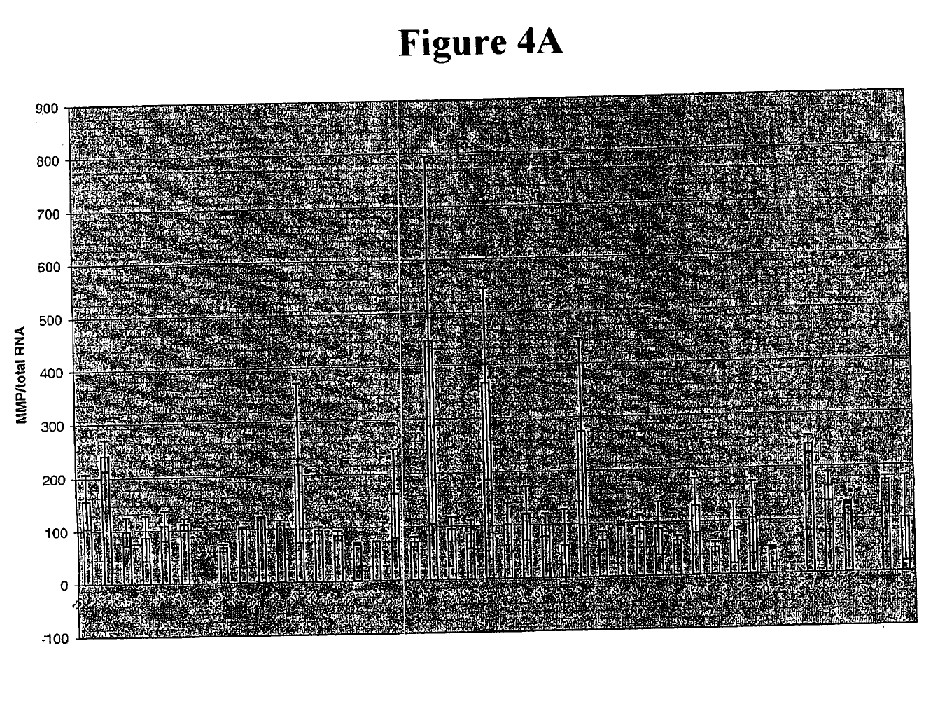

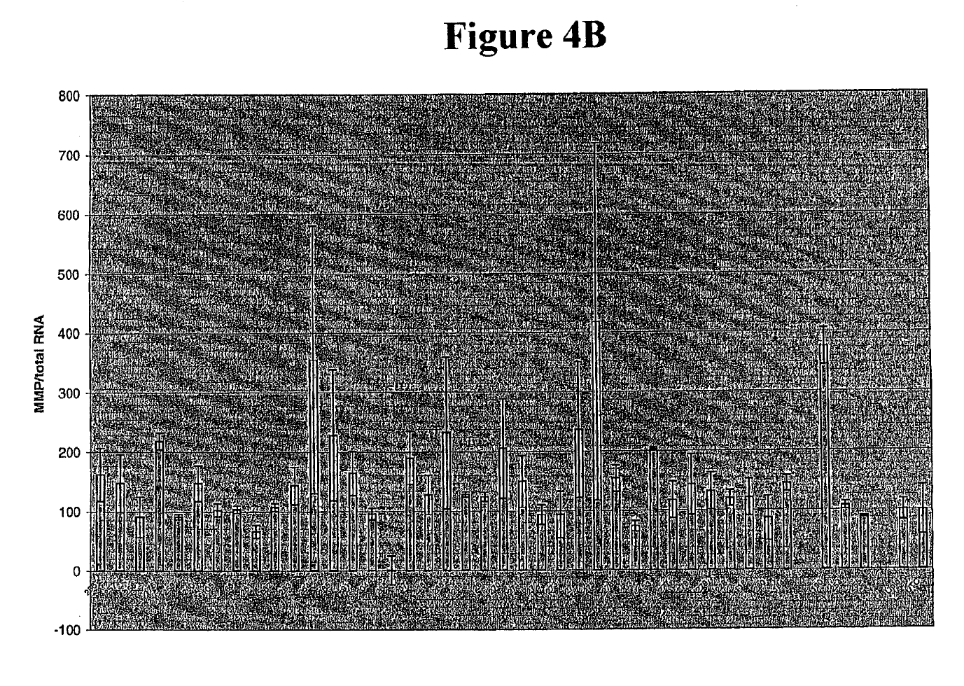

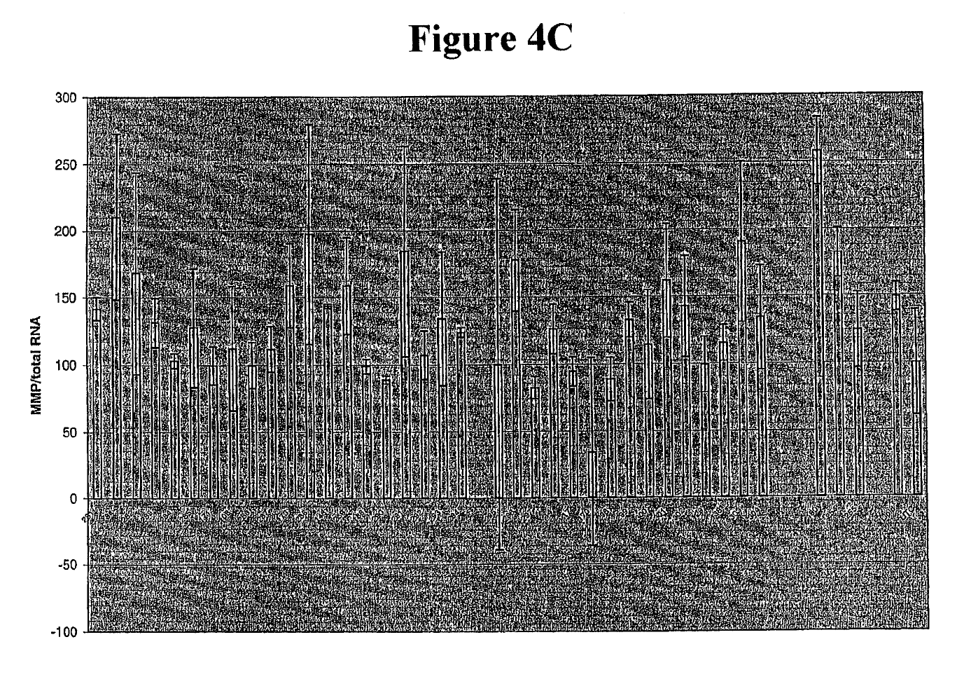

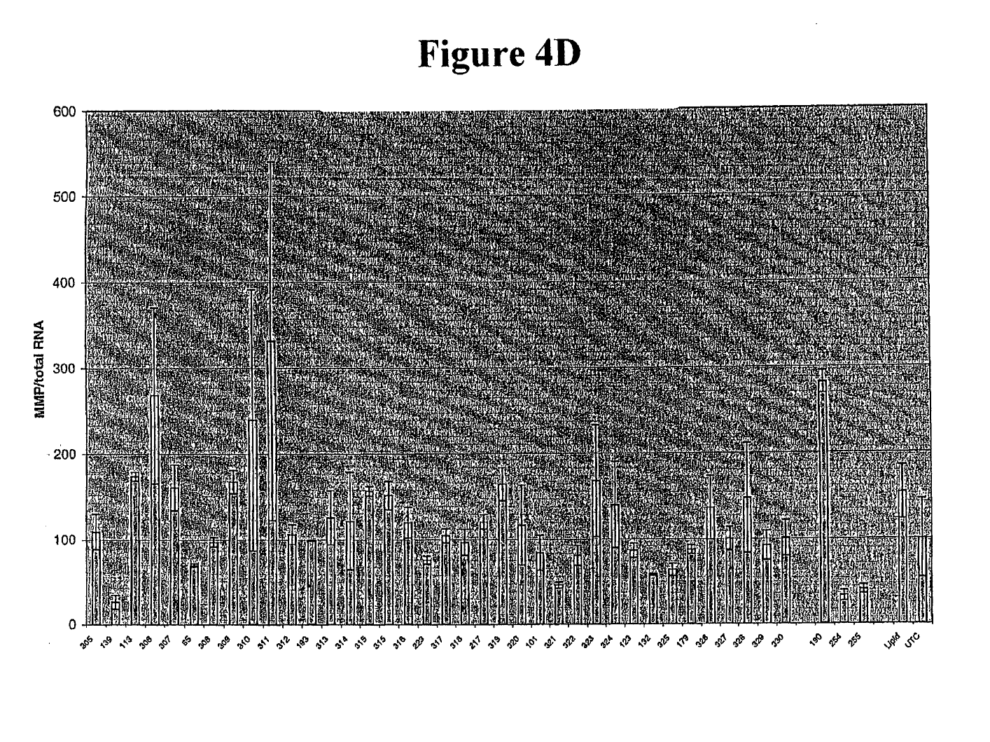

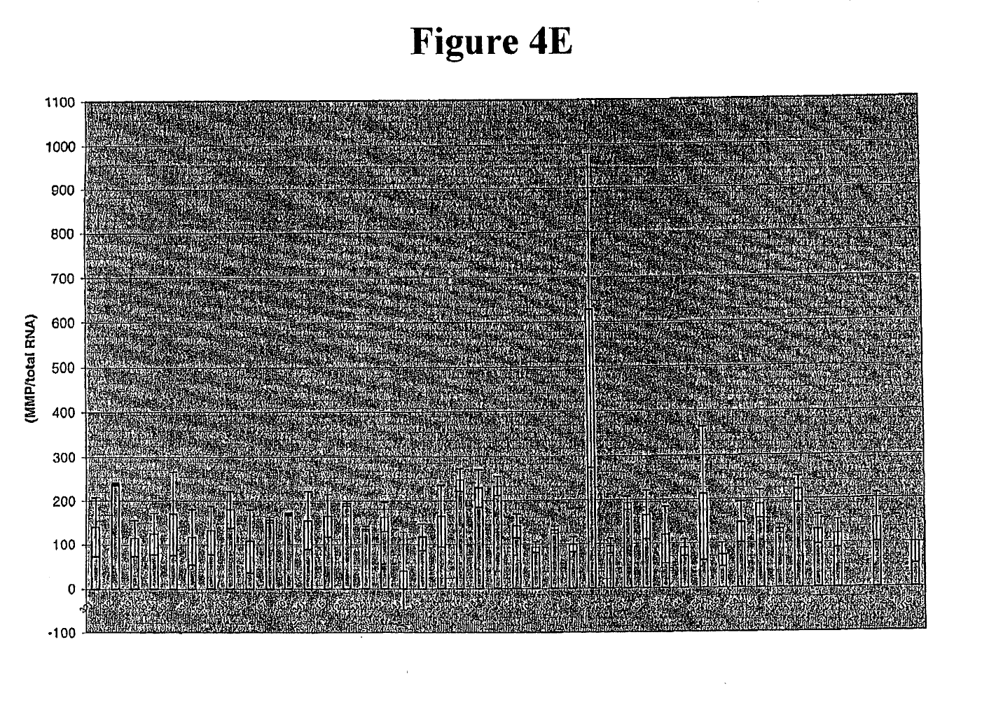

MMP assays were performed using the Enzchek Gelatinase/Collagenase Kit (Molecular Probes #E121055). HUVEC were transfected with the same antisense oligonucleotide library as in Example 14 using LIPOFECTIN™ in 96-well plates (one antisense oligonucleotide per well) with 100 μL final media volume. Forty-eight hours post-transfection, 25 μL 1×reaction buffer was added to black Corning clear-bottom plates (VWR #29444012) for UV plate reader. P-aminophenylmercuric acetate (APMA, 3.5 mg/ml in 0.1 N NaOH) solution was made immediately before use and diluted 1:4 in 1×reaction buffer, then adjusted to pH 7-8 using 1 N HCl. This solution is only stable for about one hour at 4° C. One mL of water was added to the DQ-gelatin substrate provided in the kit and incubated at 37° C. to dissolve completely. Seventy-five μL of the media was transferred to the UV plate wells. And 11 μL APMA solution was added to each well (having at least 3 wells with control media for background). Plates were incubated at 37° C. for 30 minutes and a 1:10 dilution of substrate in 1×reaction buffer was prepared. One hundred μL of diluted substrate was added to each well and plates were incubated overnight (about 18 hours) in the dark. Remaining media was removed from cells, cells were washed in 150 and 150 μL RLT (Qiagen RNeasy kit) was added to lyse cells. Total RNA was purified using Qiagen RNeasy 96 robot. MMP plates were read at 485 nm excitation/530 nm emission on an automatic plate reader. After subtracting MMP background, MMP data was normalized to total RNA level determined by Ribogreen assay (Molecular Probes #R11491). The results are summarized in FIGS. 4A-4E. Unt=untreated, 254=positive control, 190=positive control, 255=negative control, lipid=negative control. Oligonucleotides which significantly decreased MMP RNA levels include 254, 255, 139, 266, 77 and 16. Because MMPs degrade extracellular matrix and allow endothelial cells to migrate and form new vessels, these antisense oligonucleotides correspond to genes which promote angiogenesis and represent angiogenesis inhibitors. Conversely, some oligonucleotides increased MMP RNA levels, including 190, 176, 350, 347, 348, 349, 332 and 232. These oligonucleotides correspond to genes which inhibit angiogenesis, and represent potential inducers of angiogenesis.

### Example 15

Adipocyte differentiation

Increased triglyceride formation and increased leptin secretion are markers of adipocyte differentiation. To determine gene products which play a role in adipocyte differentiation, human white preadipocyte SP—F cells (2×10⁶ cells, Zen-Bio) were cultured in preadipocyte media (Zen-Bio) in a T175 flask for 3 days at 37° C. in 5% CO₂. Cells were seeded into a 96-well plate, 3×10³ cells per well in preadipocyte media, and grown to 80% confluence. Cells were transfected with 250 nM of the following antisense oligonucleotide library with 10 μl/ml Lipofectin (Gibco BRL): 358 (universal control), 210, 359, 305, 139, 113, 306, 307, 65, 308, 309, 310, 311, 312, 193, 313, 314, 315, 316, 223, 317, 318, 217, 319, 320, 101, 321, 322, 323 and 324.

Transfection was done as follows. Oligonucleotides were diluted in a 96-well plate (one oligonucleotide per well) to 500 nM with Opti-MEM (Gibco BRL) (135 μl Opti-MEM+15 μl of 5 μM oligonucleotide) and incubated at room temperature for 15 min. 150 μl of (15 ml Opti-MEM+300 μl Lipofectin) was transferred to each well of the 96-well plate, and the plate was incubated at room temperature for 15 minutes. Cells were washed once with Opti-MEM, and 100 μl of the above oligonucleotide-Lipofectin mix was added to each well in triplicate plates, and the plates were incubated at 37° C. for 4 hours. Transfection medium was removed and replaced with preadipocyte medium, and the plates were incubated for 3 days. To promote adipocyte differentiation, three days after transfection, differentiation media (DM) (Zen-Bio) plus 1 μM insulin and 0.25 mM isobutylmethylxanthine (IBMX) was added and cells were incubated for 2 days. DM was then replaced with adipocyte media (AM) (Zen-Bio) for 2 days, DM plus 1 μM insulin for 2 days and AM for 2 days.

### Example 16

Triglyceride assay

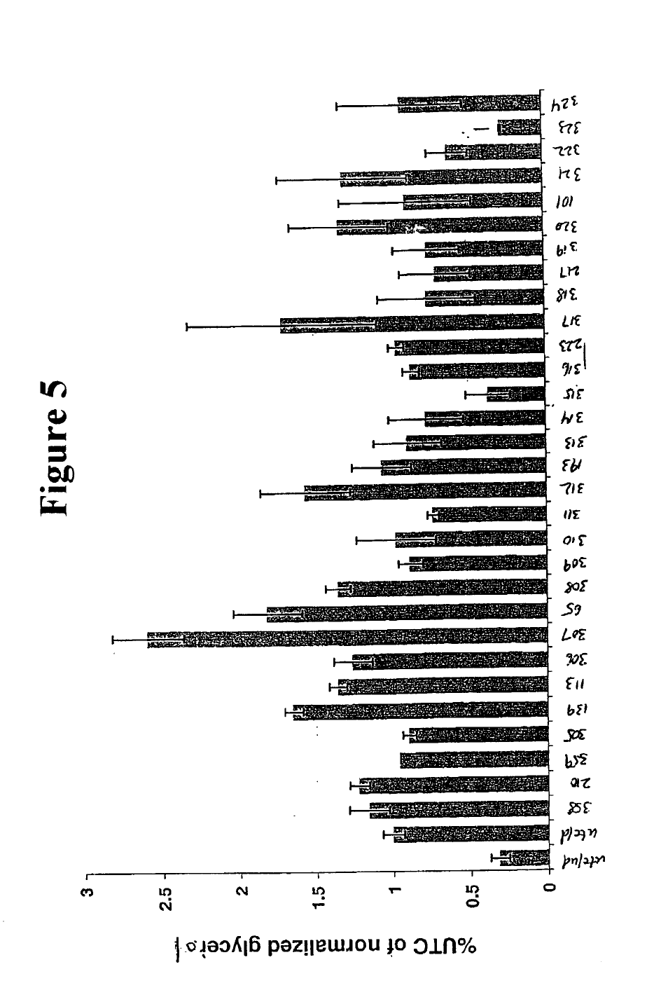

Cell media were removed from adipocytes which were differentiated as described in Example 16 (media kept for leptin assay) and cells were washed twice with PBS. Fifty μl of 0.2% IGPAL PBS was added to each well and cells were incubated for 10 min at room temperature. One hundred μl of Affinity Triglyceride assay reagent (SIGMA) was added to the resulting cell lysate in each well, and plates were incubated for 1 hour at 37° C. Plates were read in a plate reader at an OD of 515 nm. Glycerol per well was calculated by y=(x-0.06)/0.06. Five μl of cell lysate was diluted in 95 μl PBS and 10 μl of this mix was combined with 100 μl 1×protein assay buffer, then read at 450 nm as total protein. The glycerol amount was normalized by total protein (glycerol/total protein). The results are shown in FIG. 5. The two antisense oligonucleotides which inhibited triglyceride formation the most were 315 and 323. Several oligonucleotides also promoted differentiation (307, 65 and 317). These oligonucleotides correspond to gene products which play a role in adipocyte differentiation.

### Example 17

Leptin Assay

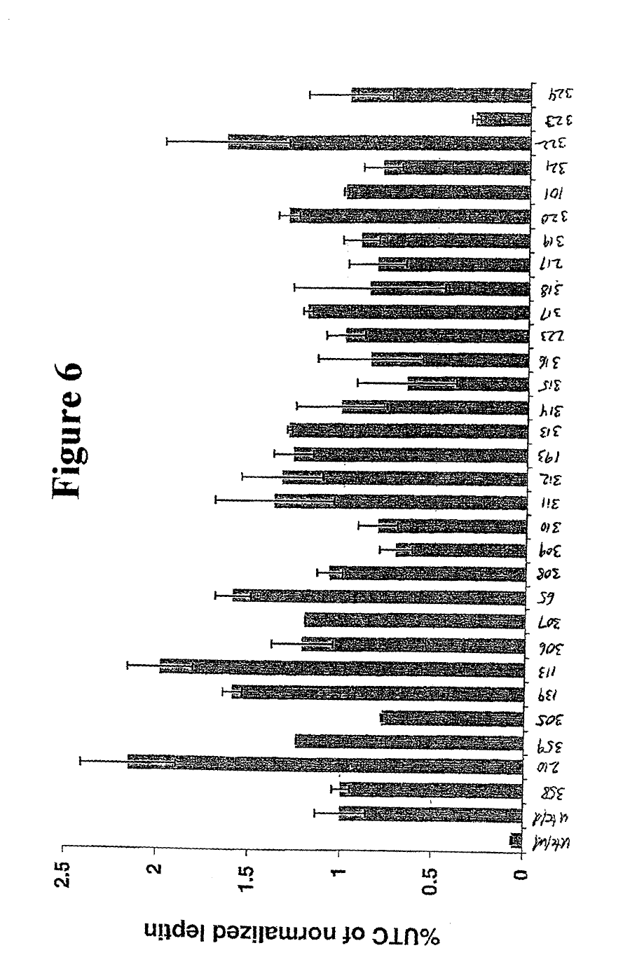

The cell media from the differentiated adipocytes of Example 17 was diluted lOx before the assay which was performed according to the R&D leptin assay kit instructions (R&D Systems, Minneapolis, Minn.). The results are shown in FIG. 6. Oligonucleotide 323 significantly inhibited leptin secretion. Several oligonucleotides also promoted leptin secretion (210, 113 and 322). These oligonucleotides correspond to gene products which play a role in adipocyte differentiation.

### Example 18

Antisense Oligonucleotide Library Screening in a fas Cross-Linking Antibody Murine Model for Hepatitis

Injection of agonistic fas-specific antibody into mice can induce massive hepatocyte apoptosis and liver hemorrhage, and death from acute hepatic failure (Ogasawara, J., et al., Nature 364:806-809, 1993). Apoptosis-mediated aberrant cell death has been shown to play an important role in a number of human diseases. For example, in hepatitis, fas and fas ligand up-regulated expression are correlated with liver damage and apoptosis. It is thought that apoptosis in the livers of patients with fulminant hepatitis, acute and chronic viral hepatitis, autoimmune hepatitis, as well as chemical or drug induced liver intoxication may result from fas activation on hepatocytes. 8-10 week old female Balb/c mice are intraperitoneally injected with different antisense oligonucleotides (one per mouse), such as those shown in Tables 1A-1C, daily for 4 days. Four hours after the last dose, 7.5 μg of mouse fas antibody (Pharmingen, San Diego, Calif.) is injected into the mice. Mortality of the mice is measured for more than 10 days following antibody treatment. Oligonucleotides which protect the fas antibody treated mice from death correspond to genes involved in fas-specific antibody-mediated death from acute hepatic failure. Saline or scrambled control oligonucleotide had no protective effect.

### Example 19

Identification of Genes Involved in Phorbol Myristate Acetate (PMA)-Induced L-Selectin Shedding

Genes involved in promoting PMA-induced L-selectin shedding are determined in Jurkat T cells. Jurkat cells are electroporated with 20 μM of each oligonucleotide from an antisense oligonucleotide library. 24 hours after oligonucleotide treatment, L-selectin shedding is induced with 100 nM PMA (Calbiochem, San Diego, Calif.) for 5 minutes at 37° C. L-selectin cell surface expression is analyzed by flow cytometry using a FACScan (Becton Dickinson, San Jose, Calif.). Antisense oligonucleotides which inhibit L-selectin shedding correspond to genes which promote L-selectin shedding.

### Example 20

Mouse Experimental Autoimmune Encephalomyelitis (EAE) Model for Multiple Sclerosis

Experimental autoimmune encephalomyelitis (EAE) is an inflammatory, demyelinating central nervous system disease frequently used as an animal model for multiple sclerosis. It is inducible in genetically susceptible animals by immunization with whole spinal cord homogenate or protein components of the myelin sheath such as myelin basic protein (MBP) or proteolipid protein (PLP), or by transfer of MBP- or PLP— specific T cells. Myers et al., J. Immunol. 1993, 151, 2252-2260.

CSJLF-1 mice (Jackson Laboratory, Bar Harbor, Me.) are immunized with the pl3 peptide (Research Genetics, Huntsville, Ala.) which corresponds to residues 139-151 of PLP and is encephalitogenic in these mice. Mice are immunized essentially as described in Myers et al., (J. Neuroimmunology 1992, 41, 1-8). Briefly, mice are injected in the hind footpads and the base of the tail with 50-100 ug of pl3 peptide, emulsified in CFA (Difco, Detroit, Mich.) fortified with 4 mg/ml of heat-killed H37Ra Mycobacterium tuberculosis bacteria (Difco). At the time of footpad injections and again 2 days later, mice are also injected intravenously with 500 ng of pertussis toxin (Sigma, St. Louis, Mo.). Mice are treated with different antisense oligonucleotides at various doses, beginning one day before pl3 immunization except where indicated otherwise. Oligonucleotides are formulated in 0.9% saline and are administered daily by subcutaneous injection (one per mouse), with dosing continuing until more than 50% of the p13-immunized but saline-treated control group begin showing symptoms of disease. Dosing is then terminated and mice are observed for effects of treatment on the course of disease. Disease severity is scored on a scale of 0 to 5 with 0=no symptoms, 1 flaccid tail, 2=hind limb weakness, 3=hind limb paralysis, 4=hind and front limb paralysis and 5=moribund or dead. Time until disease onset is also measured and compared to pl3-immunized control mice that receive saline instead of oligonucleotide. Oligonucleotides which produce disease with a severity score lower than that of the saline-treated control group correpond to genes involved in EAE and potentially involved in human MS.

### Example 21

Mouse Collagen-Induced Arthritis (CIA) Model for Rheumatoid Arthritis

A model for human rheumatoid arthritis has been developed wherein mice are immunized with bovine type II collagen. Anderson et al., J. Immunol. 1991 147, 1189-1193, citing Trentham et al., J. Exp. Med 1977, 146, 857. Swelling and inflammation of the joints follows in approximately 3 weeks, with joint distortion and ankylosis typical of rheumatoid arthritis. This model has been used to study the effects of the antisense oligonucleotide 17044, targeted to mouse integrin a4, on arthritis in mice.

DBA/1LacJ mice aged 6 to 8 weeks are used and assigned to groups, ten mice per group. On day 0 mice are immunized at the base of the tail with 100 ug of bovine type II collagen which was emulsified in Complete Freund's Adjuvant (CFA). On day 7, a second booster dose of collagen is administered by the same route. On day 14 the mice are injected subcutaneously with 100 ug of lipopolysaccharide (LPS). Weights are recorded weekly. Mice are inspected daily for the onset of CIA, which is characterized by erythema and edema. Upon the onset of the disease, paw widths and rear ankle widths of affected and unaffected joints are measured three times a week using a constant tension caliper. In addition, limbs are clinically evaluated and graded 0-4, where 0=normal; 1=one digit swollen; 2=inflammation present in more than one digit; 3=joint distortion with or without inflammation; and 4=ankylosis, detected by joint manipulation. The progression of all measurements was recorded to day 50. At the end of the observation period for each mouse, all paws re removed and examined histologically.

Antisense oligonucleotide libraries, the positive control drug (cyclophosphamide, 5 mg/kg), and the vehicle are administered daily to each mouse intraperitoneally (IP) (one antisense oligonucleotide per mouse) starting on day −3 and continuing for the duration of the study. Each animal receives 10 mg/kg as a bolus daily dose. Oligonucleotides which reduce arthritis incidence correspond to genes involved in collagen-induced arthritis.

### Example 22

Determination of Genes Involved in Expression of Cell Adhesion Molecules in HUVEC

Expression of the cells adhesion molecules intercellular adhesion molecule-1 (ICAM-1), vascular cell adhesion molecule-1 (VCAM-1) and endothelial cell adhesion molecule-1 (ELAM-1) can be quantitated using specific monoclonal antibodies in an ELISA. Human HUVEC are grown to confluence in 96 well microtiter plates and pretreated with an antisense oligonucleotide library (one antisense oligonucleotide per well). Cells are then stimulated with either IL-1β or TNF-α for 4 to 8 hours to quantitate ELAM-1, or 8 to 24 hours to quantitate ICAM-1 and VCAM-1. Following cytokine incubation, cells are gently washed three times with PBS, then directly fixed on the microtiter plate with 1 to 2% paraformaldehyde diluted in PBS for 20 minutes at 25° C. Cells are washed again with PBS three times. Nonspecific binding sites are blocked with 2% bovine serum albumin in PBS for 1 hour at 37° C. Cells are incubated with the appropriate monoclonal antibody diluted in blocking solution for 1 hour at 37° C. Unbound antibody is removed by washing the cells three times with PBS. Antibody bound to the cells is deteted by incubation with a 1:1000 dilution of biotinylated goat anti-mouse IgG in blocking solution for 1 hour at 37° C. Cells are washed three times with PBS then incubated with a 1:1000 dillution of streptavidin conjugated to β-galactosidase for 1 hour at 37° C. Cells are washed three times with PBS for 5 minutes each. The amount of β-galactosidase bound to the specific monoclonal antibody is determined by developing the plate in a solution of 3.3 mM chlorophenol red-β-D-galactopyranoside, 50 mM sodium phosphate, 1.5 mM MgCl₂, pH 7.2 for 2 to 15 minutes at 37° C. The concentration of the product is determined by measuring the absorbance at 575 nm in a plate reader. Oligonucleotides which inhibit cytokine-stimulated expression of cell adhesion molecules correspond to genes involved in this process.

What is claimed is:

1. A method for identifying one or more genes involved in a response by a cell, tissue or organism to a stimulus, comprising the steps of:
a.) contacting cells, tissues or organisms which are capable of exhibiting a particular response to said stimulus with a library of antisense oligonucleotides prior to treatment with said stimulus; and
b.) determining which antisense oligonucleotides within said library modulate said response, wherein antisense oligonucleotides which modulate said response correspond to gene products involved in said response.

2. The method of claim 1, wherein said cells are divided into one or more substantially identical subpopulations prior to contacting with said library of oligonucleotides, wherein each subpopulation is contacted with one member of said library of antisense oligonucleotides.

3. The method of claim 1, wherein said compound is a cytokine or growth factor.

4. The method of claim 3, wherein said cytokine or growth factor is TNF-α, IL-1 or IFN-γ.

5. The method of claim 1, wherein said response is secretion of a compound.

6. The method of claim 5, wherein said compound is a cytokine or growth factor.

7. The method of claim 1, wherein said response is modulation of expression of a cell surface protein.

8. The method of claim 7, wherein said cell surface protein is a cell adhesion protein.

9. The method of claim 1, wherein said response is modulation of inflammation.

10. The method of claim 1, wherein said response is inhibited.

11. The method of claim 1, wherein said response is stimulated.

12. The method of claim 1, wherein said response is a modulation of apoptosis or cell cycle profile.

13. The method of claim 1, wherein said response is modulation of angiogenesis.

14. The method of claim 1, wherein said response is modulation of insulin signaling, glycogenolysis or adipocyte differentiation.

15. A method for identifying one or more genes involved in a phenotype of a cell, tissue or organism, comprising the steps of:
a.) contacting one or more substantially identical subpopulations of said cell, tissue or organism which exhibits said phenotype with a library of antisense oligonucleotides, wherein each subpopulation is contacted with one member of said library of antisense oligonucleotides; and
b.) performing a primary phenotypic assay to determine which antisense oligonucleotides within said library modulate said phenotype, wherein antisense oligonucleotides which modulate said phenotype correspond to genes involved in said phenotype.

16. The method of claim 15, wherein said phenotype is associated with a disease state.

17. The method of claim 15, wherein said disease state is cancer, undesired angiogenesis, inflammation or a metabolic disorder.

18. The method of claim 17, wherein said metabolic disorder is diabetes.

19. The method of claim 15, further comprising the step of performing a secondary phenotypic assay.

20. The method of claim 19, wheren said secondary phenotypic assay is a low density array.

21. The method of claim 19, further comprising the step of performing a tertiary phenotpic assay.

22. The method of claim 21, wherein said tertiary phenotypic assay is a high density array.

23. A method for identifying genes expressed in dendritic cells that regulate co-stimulation of T-cells, comprising the steps of:
a.) culturing dendritic cells in the presence of one or more cytokines to activate said dendritic cells;
b.) contacting one or more substantially identical subpopulations of said activated dendritic cells with a library of antisense oligonucleotides, wherein each subpopulation is contacted with one member of said library;
c.) adding T-cells to said antisense oligonucleotide-treated activated dendritic cells; and
d.) measuring IL-2 production, wherein antisense oligonucleotides which modulate IL-2 production correspond to genes which play a role in co-stimulation of T cells.

24. The method of claim 23, wherein said cytokines comprise IL-4 and GM-CSF.

25. The method of claim 23, wherein said antisense oligonucleotide inhibits production of IL-2.

26. A method for identifying genes that play a role in T cell-mediated inflammation, comprising the steps of:
a.) culturing dendritic cells in the presence of one or more cytokines to activate said dendritic cells;
b.) contacting one or more substantially identical subpopulations of said activated dendritic cells with a library of antisense oligonucleotides, wherein each subpopulation is contacted with one member of said library;
c.) adding T-cells to said antisense oligonucleotide-treated activated dendritic cells; and
d.) measuring IL-2 production, wherein antisense oligonucleotides which inhibit IL-2 production correspond to genes which increase inflammation.

27. The method of claim 26, wherein said cytokines comprise IL-4 and GM-CSF.

28. The method of claim 26, further comprising the step of adding a CTLA4-Ig fusion protein after treatment with antisense oligonucleotide.

29. A library comprising between about 10 and 10,000 prevalidated antisense oligonucleotides.
